(19) United States
(12) Patent Application Publication
(10) Pub. No.: US 2016/0143739 A1
(43) Pub. Date: May 26, 2016

(54) PROSTHETIC VENTRICULAR HEART SYSTEM

(71) Applicant: BOSTON SCIENTIFIC SCIMED INC., MAPLE GROVE, MN (US)

(72) Inventors: Fergal Horgan, Co. Mayo (IE); Martyn G MR Folan, Co. Galway (IE); Patrick Connolly, Galway (IE); Marie Turkington, Co. Mayo (IE)

(73) Assignee: BOSTON SCIENTIFIC SCIMED INC., MAPLE GROVE, MN (US)

(21) Appl. No.: 14/950,637

(22) Filed: Nov. 24, 2015

Related U.S. Application Data
(60) Provisional application No. 62/084,215, filed on Nov. 25, 2014.

(51) Int. Cl. A61F 2/24; A61M 1/10

(52) U.S. Cl. CPC A61F 2/2487; A61M 1/1053; A61F 2220/0008

(57) ABSTRACT

An implantable heart system for the treatment of a heart with impaired function. The implantable heart system includes a prosthetic heart chamber assembly and a cooperative mechanism. The prosthetic heart chamber assembly comprises a first prosthesis, a second prosthesis, and a prosthetic heart chamber interconnecting the first and second prostheses. One of the prostheses is a valve and the other prosthesis is either a valve or a tubular prosthesis. The cooperative mechanism is selected from the group consisting of an adhesive; an anchor; a filler material; a shape memory element; an expandable member; a pumping agent; material attached to, or forming a part of, the wall of the prosthetic heart chamber; and combinations thereof.

### CROSS-REFERENCE TO RELATED APPLICATIONS

This application claims the benefit of U.S. Provisional Application No. 62/084,215, filed Nov. 25, 2014.

### STATEMENT REGARDING FEDERALLY SPONSORED RESEARCH

Not Applicable

### BACKGROUND

Heart failure is a significant public health issue. Epidemiological surveys using clinical findings suggest that between 1-2% of western adult populations are affected by heart failure. Further evidence from the United States shows that in the last twenty years there has been a fourfold increase in unadjusted mortality rates for heart failure. The most obvious reason for this increase mortality rate is the increase in the aging populations, although changes in classification may be another factor. Prevalence is also increasing because more people are surviving myocardial infarcts (MIs).

In general terms, heart failure occurs as a result of damage to the heart that may be caused by acquired or congenital circulatory or myocardial disorders. In the majority of cases, heart failure occurs due to ischemic heart disease and MI-induced damage to the heart muscle. Other disorders that may lead to the onset of heart failure include cardiac dysrhythmias, chronic hypertension; dilated, hypertrophic, or restrictive cardiomyopathy; severe valvular disorders; congenital heart defects; and myocardial or valvular infections. In younger patients, heart failure may be caused by anemia, chronic renal and pulmonary diseases, or hyperthyroidism.

In the cases where the patient survives an acute episode of MI, a process of left ventricular remodeling is initiated, with further architectural and structural changes to the ventricle. Remodeling occurs in both the infarcted and remaining non-infarcted regions, further contributing to the ventricular dysfunction. The extent of ventricular dysfunction depends on the size and location of the infarct, the presence of previous infarcts elsewhere in the heart, the remaining coronary supply with or without collaterals, and the involvement of other cardiac structures which influence ventricular function, such as the conducting tissue, heart valves, and pericardium.

Specifically, the region of necrosis involves damaged myocytes and disruption of extracellular matrix. Loss of type I collagen fibers and intermyocyte collagen struts occurs due to activation of matrix metalloproteineases (1, 2, and 9 predominate in the heart), and is replaced by a deposition of collagen II and IV from fibroblasts stimulated by aldosterone and angiotension II. There is an overall increase in the myocardial collagen content from 5% up to 25%, but it is laid down in an irregular fashion, which disrupts the fine myocardial architecture. This allows myocyte slippage in the longitudinal direction, leading to the loss of cells and vasculature to infarct thinning and expansion. This is more extensive in areas with complete absence of blood supply. The presence of collaterals, or late revascularization of the culprit vessel, reduces infarct expansion. Anterior infarcts lead to an increase in left ventricular circumference. This expansion alters the geometry of the left ventricle, with the normal ellipsoid shape progressively replaced by a more spherical shape. This sphericity leads to a subsequent reduction in efficiency of blood ejection from heart chamber, higher filling pressures, and reduced exercise capacity.

The infarction of one region of the left ventricular wall requires the remaining myocardium to compensate mechanically in order to maintain adequate cardiac output. Eccentric hypertrophy with sarcomeric replication in series occurs, which results in further increases in ventricular dimensions and compliance. The increased wall stress may stimulate the remaining non-infarcted myocardium to hypertrophy in a concentric manner, most commonly seen at the border zone of the infarct. This progress starts 1-2 months after the initial infarction, and may progress for years unless a terminal cardiac event intervenes.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the disclosure and/or additional embodiments of the disclosure may be found in the Detailed Description below.

### BRIEF SUMMARY

A prosthetic heart chamber assembly comprising a first prosthesis, a second prosthesis, and a prosthetic heart chamber interconnecting the first and second prostheses.

The first prosthesis may be a valve or a tubular prosthesis.

The tubular prosthesis may be selected from the group consisting of a stent, a stent-graft, and a graft.

The first prosthesis may a tubular prosthesis or a valve.

The first prosthesis may be self-expanding.

The first prosthesis may have a cover attached to the outer surface.

The prosthetic heart chamber assembly may include a means for anchoring the first prosthesis (an anchoring means).

The second prosthesis may be a valve.

The second prosthesis may have a cover attached to the outer surface.

The prosthetic heart chamber assembly may include a means for anchoring the second prosthesis (an anchoring means).

The anchoring means may be selected from the group consisting of: barbs; spikes; anchors; adhesion elements; raised elements or regions of the prosthesis; enlarged diameter regions of the prosthesis; means for controlled tissue ingrowth; and combinations thereof.

The anchoring means may extend through a cover positioned on the outer surface of the prosthesis.

The anchoring means may be attached to the outer surface of a cover positioned on the outer surface of the prosthesis.

The prosthetic heart chamber assembly may have a cooperative mechanism compartment.

The prosthetic heart chamber assembly may form a part of an implantable heart system.

The heart system may comprise a cooperative mechanism associated with the prosthetic heart chamber assembly.

The heart system may be constructed and arranged to treat a heart with impaired function.

The heart system may be constructed and arranged to treat a damaged heart (e.g. heart failure).

The heart system may be constructed and arranged to slow or stop the progression of damage to the heart (e.g. slow/stop heart failure).

The heart system may be constructed and arranged to facilitate the pumping of blood through the heart (facilitate the function of systole).

The heart system may be constructed and arranged to isolate/partition a damaged section of a native heart chamber wall.

The heart system may be constructed and arranged to prevent further dilation, enlargement, or remodeling of the heart.

The heart system may be constructed and arranged to change the volume of the native heart chamber.

The heart system may be constructed and arranged to reduce the volume of blood to be ejected.

The heart system may be constructed and arranged to reduce resistance to blood flow.

The heart system may be constructed and arranged to change the shape of the native heart chamber.

The heart system may be constructed and arranged to reduce the amount of pressure or stress on the native heart chamber wall.

The heart system may be constructed and arranged to provide the heart with an opportunity to recuperate from damage.

The heart system may be constructed and arranged to compensate for stiffness of the native heart chamber wall.

The cooperative mechanism may be constructed and arranged to anchor the prosthetic heart chamber.

The cooperative mechanism constructed and arranged to anchor the prosthetic heart chamber may be an anchor; an adhesive; and combinations thereof.

The cooperative mechanism may be a plurality of cooperative mechanisms.

The cooperative mechanism may be secured to the prosthetic heart chamber assembly.

The cooperative mechanism may be secured to the prosthetic heart assembly by an adhesive.

The cooperative mechanism may be positioned inside the cooperative mechanism compartment of the prosthetic heart chamber

The cooperative mechanism may be selected from the group consisting of an adhesive; an anchor; a filler material; a shape memory element; an expandable member; a pump device; a material attached to, or forming a part of, the wall of the prosthetic heart chamber; the cooperative mechanism compartment; and combinations thereof.

The material attached to, or forming a part of, the wall of the prosthetic heart chamber may be selected from the group consisting of an elastic material; a non-elastic material; an electric electroactive polymer; and combinations thereof.

The material attached to, or forming a part of, the wall of the prosthetic heart chamber may comprise an elastic material.

The material attached to, or forming a part of, the wall of the prosthetic heart chamber may include an elastic region and a non-elastic region.

The material attached to, or forming a part of, the wall of the prosthetic heart chamber may be an electric electroactive polymer. An electric field generating device may be in electrical communication with the prosthetic heart chamber assembly.

The electric electroactive polymer may mechanically facilitate the prosthetic heart chamber in the function of systole by having a first shape when the heart is in diastole and a second, different, shape when the heart is in systole, where the second shape is constructed and arranged to aid in the ejection of blood from the prosthetic heart chamber.

The cooperative mechanism may stretch a portion of the prosthetic heart chamber taut. The portion of the prosthetic heart chamber stretched taut by the anchors may comprise an elastic material.

The cooperative mechanism may be filler material.

The filler material may mechanically facilitate the prosthetic heart chamber in the function of systole by reducing the volume of blood to be ejected from the prosthetic heart chamber.

The filler material may mechanically facilitate the prosthetic heart chamber in the function of systole by reducing resistance to blood flow through the prosthetic heart chamber.

The cooperative mechanism may be a shape memory element.

The shape memory element may mechanically facilitate the prosthetic heart chamber in the function of systole due to a reversible change in the shape of the shape memory element.

The cooperative mechanism may be an expandable member.

The expandable member may be a balloon or the cooperative mechanism compartment.

The expandable member may mechanically facilitate the prosthetic heart chamber in the function of systole by expanding to an enlarged state.

The cooperative mechanism may be a pump device.

The pump device may mechanically facilitate the prosthetic heart chamber in the function of systole by expanding a cooperative mechanism compartment or by expanding an expandable member.

The pump device may be in fluid communication with the cooperative mechanism compartment. The pump device may be in fluid communication with the cooperative mechanism compartment by means of an elongated hollow tube.

The cooperative mechanism compartment may include a section of material comprising an elastic material.

The pump device may be in fluid communication with the expandable member. The pump device may be in fluid communication with the expandable member by means of an elongated hollow tube.

The cooperative mechanism may comprise a first cooperative mechanism and a second cooperative mechanism different than the first cooperative mechanism.

The first cooperative mechanism may be constructed and arranged to anchor the prosthetic heart chamber.

The first cooperative mechanism may be an anchor; an adhesive; and combinations thereof.

The first cooperative mechanism may be an adhesive and the second cooperative mechanism may be a filler material.

The first cooperative mechanism may be a filler material and the second cooperative mechanism may be an anchor.

The first cooperative mechanism may be a shape memory element and the second cooperative mechanism may be an anchor.

The first cooperative mechanism may be a shape memory element and the second cooperative mechanism may be adhesive.

The first cooperative mechanism may be a balloon and the second cooperative mechanism may be an adhesive.

The cooperative mechanism may comprise a first cooperative mechanism, a second cooperative mechanism, and a third cooperative mechanism, the first, second, and third cooperative mechanisms being different cooperative mechanisms.

The first cooperative mechanism may be an anchor; the second cooperative mechanism may be an expandable member; and the third cooperative mechanism may be a pump device.

The first cooperative mechanism may be an adhesive; the second cooperative mechanism may be an expandable member; and the third cooperative mechanism may be a pump device.

The first cooperative mechanism may be an anchor; the second cooperative mechanism may be a cooperative mechanism compartment; and the third cooperative mechanism may be a pump device.

The first cooperative mechanism may be an adhesive; the second cooperative mechanism may be a cooperative mechanism compartment; and the third cooperative mechanism may be a pump device.

These and other embodiments which characterize the disclosure are pointed out with particularity in the claims annexed hereto and forming a part hereof. However, for further understanding of the disclosure, reference can be made to the drawings which form a further part hereof and the accompanying descriptive matter, in which there is illustrated and described embodiments of the disclosure.

### BRIEF DESCRIPTION OF THE SEVERAL VIEWS OF THE DRAWINGS

A detailed description of the disclosure is hereafter described with specific reference being made to the drawings.

FIG. 1 is partial cross-sectional view of a heart.

FIG. 2A shows blood volume in the left atrium being retained by a closed mitral valve.

FIG. 2B shows systole contraction of the left atrium with a closed aortic valve.

FIG. 2C shows systole contraction of the left ventricle with a closed mitral valve and an open aortic valve.

FIG. 3A shows an embodiment of a prosthetic heart chamber assembly.

FIG. 3B shows an embodiment of a prosthetic heart chamber assembly.

FIG. 4 shows an embodiment of a prosthetic heart chamber assembly.

FIG. 5 shows an example of cooperative mechanisms for use with a prosthetic heart chamber assembly. For simplicity, in the embodiments shown in FIGS. 5-21, many structures of the prosthetic heart chamber assembly are omitted (e.g. the first and second prostheses) or shown schematically (e.g. the prosthetic heart chamber).

FIG. 6 shows an example of cooperative mechanisms for use with a prosthetic heart chamber assembly.

FIG. 7 shows an example of cooperative mechanisms for use with a prosthetic heart chamber assembly.

FIG. 8 shows an example of cooperative mechanisms for use with a prosthetic heart chamber assembly.

FIG. 9 shows an example of cooperative mechanisms for use with a prosthetic heart chamber assembly.

FIG. 10 shows an example of cooperative mechanisms for use with a prosthetic heart chamber assembly.

FIG. 11 shows an example of cooperative mechanisms for use with a prosthetic heart chamber assembly.

FIG. 12 shows an example of cooperative mechanisms for use with a prosthetic heart chamber assembly.

FIG. 13 shows an example of cooperative mechanisms for use with a prosthetic heart chamber assembly.

FIG. 14 shows an example of cooperative mechanisms for use with a prosthetic heart chamber assembly.

FIG. 15 shows an example of cooperative mechanisms for use with a prosthetic heart chamber assembly.

FIGS. 16A-16B show an example of cooperative mechanisms for use with a prosthetic heart chamber assembly.

FIG. 17 shows an example of cooperative mechanisms for use with a prosthetic heart chamber assembly.

FIGS. 18A-18B show an example of cooperative mechanisms for use with a prosthetic heart chamber assembly.

FIG. 19 shows an example of cooperative mechanisms for use with a prosthetic heart chamber assembly.

FIG. 20 shows an example of cooperative mechanisms for use with a prosthetic heart chamber assembly.

FIG. 21 shows an example of cooperative mechanisms for use with a prosthetic heart chamber assembly.

FIG. 22 shows a PRIOR ART prosthetic ventricular device suitable for a prosthetic chamber assembly of a heart system.

### DETAILED DESCRIPTION

While this disclosure may be embodied in many different forms, there are described in detail herein specific embodiments of the disclosure. This description is an exemplification of the principles of the disclosure and is not intended to limit the invention to the particular embodiments illustrated.

As used in this application, the terms “connect” or “engage” do not include “indirect” connection or engagement. Any suitable means can be used to connect the prosthetic heart chamber 108 to the prostheses 104, 106.

For the purposes of this disclosure, like reference numerals in the figures shall refer to like features unless otherwise indicated.

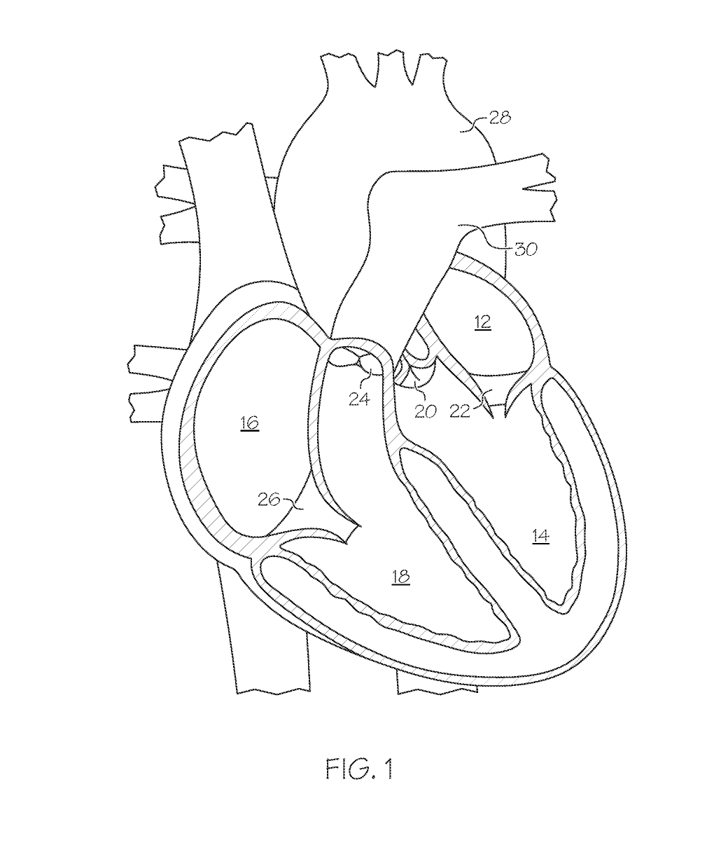

FIG. 1 is a cross-sectional view of a heart 10 that has four chambers, a left atrium 12, a left ventricle 14, a right atrium 16 and the right ventricle 18. The heart 10 has four valves, an aortic valve 20, a mitral valve 22, a pulmonary valve 24, and a tricuspid valve 26. In the left side of the heart 10, blood flows from the left atrium 12 to the left ventricle 14, and then exits the heart 10 through the aorta 28. In the right side of the heart 10, blood flows from the right atrium 16 to the right ventricle 18 and out of the heart 10 through the pulmonary artery 30.

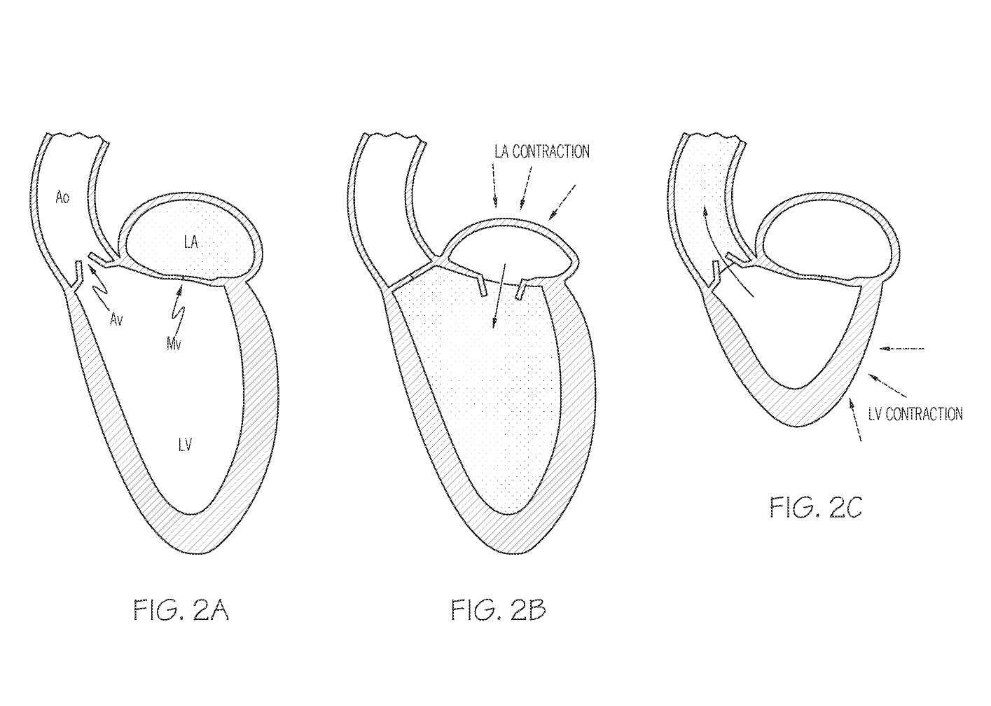

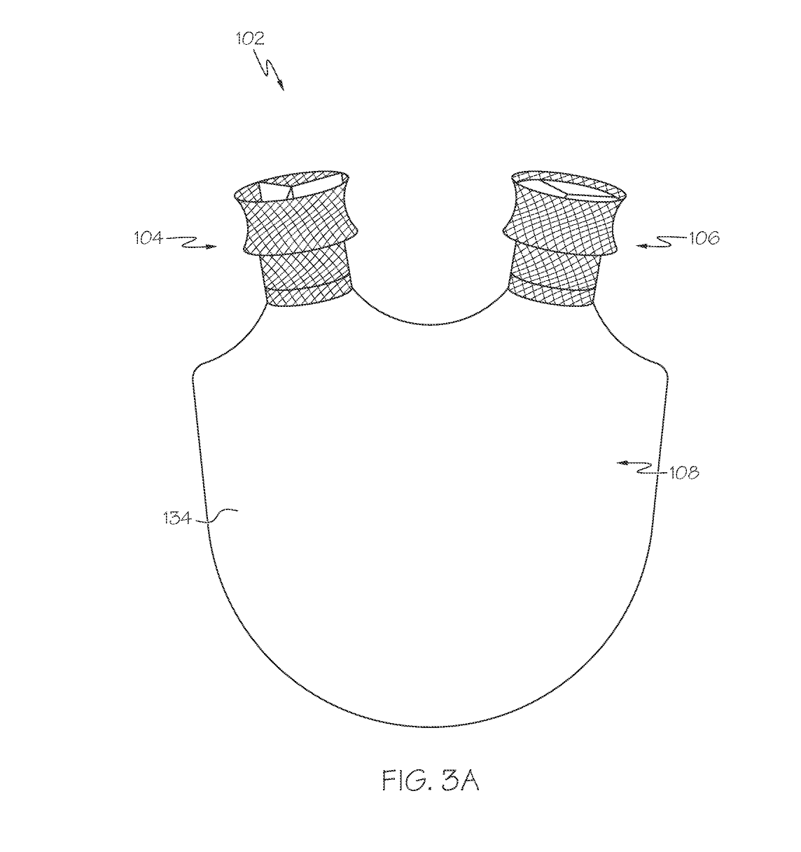

FIGS. 2A-C show how blood flows through the left chambers of the heart. FIG. 2A shows blood volume in the left atrium 12 being retained by a closed mitral valve 22. FIG. 2B shows systole contraction (arrows) of the left atrium 12 with a closed aortic valve 20, which pumps blood into the left ventricle 14. FIG. 2C shows systole contraction (arrows) of the left ventricle 14 with a closed mitral valve 22 and an open aortic valve 20, which pumps blood out of the left ventricle 14 into the aorta 28. The period of time when the heart chamber refills with blood after systole is diastole.

20

### I. Prosthetic Heart Chamber Assembly

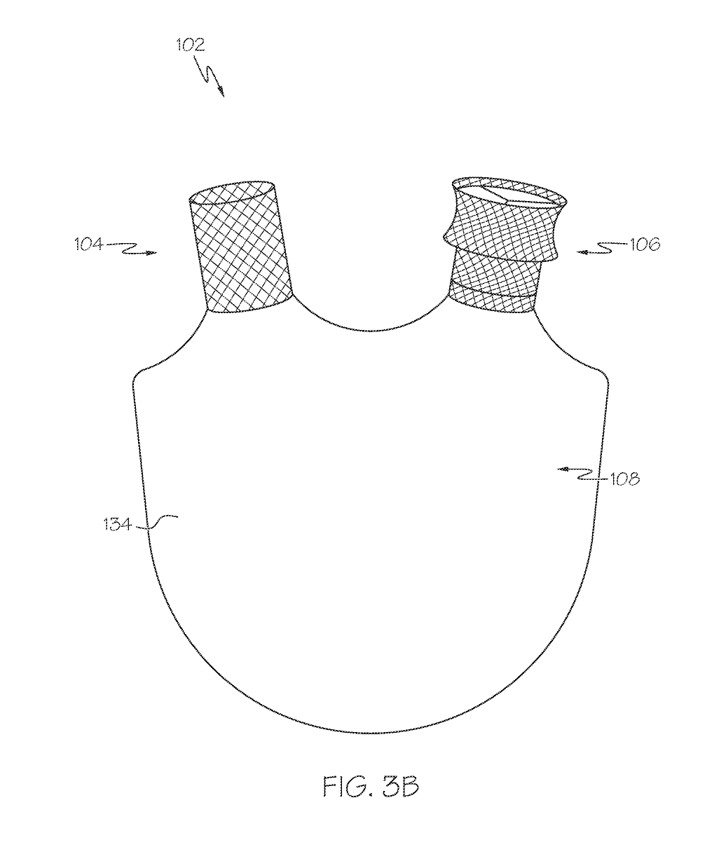

The prosthetic heart chamber assembly 102 has a first prosthesis 104, a second prosthesis 106 and a prosthetic heart chamber 108 connecting the first and second prostheses 104, 106 (see FIGS. 3A-B). The first and second prostheses 104, 106 are expandable from a reduced diameter delivery configuration to an enlarged diameter deployed configuration. A “prosthesis” as used herein may provide regulated or unregulated flow through the lumen. An example of a prosthesis that regulates flow through the lumen is a valve. Examples of prostheses that do not regulate flow through the lumen include stents, stent-grafts, and grafts. The prostheses 104, 106 may have a cover attached to an outer surface.

The first prosthesis 104 may be a valve or a tubular prosthesis. A “tubular prosthesis” as used herein is an expandable prosthesis that provides for unregulated flow through the prosthesis lumen. Thus a valve, a prosthesis that regulates flow through the lumen, is not a tubular prosthesis as used herein. Stents, stent-grafts, and grafts are examples of prostheses suitable for use as a tubular prosthesis of the prosthetic heart chamber assembly 102. The second prosthesis 104 may be a valve.

A prosthetic heart chamber assembly 102 may have a valve for the first prosthesis 104 and for the second prostheses 106 (see e.g. FIG. 3A). When a prosthetic heart assembly 102 with two valves is implanted in the left ventricle, one valve may replace the mitral valve and the other valve may replace the aortic valve.

A prosthetic heart chamber assembly 102 may have a tubular prosthesis and a valve for both of the first and second prostheses 104, 106 (see e.g. FIG. 3B). When a prosthetic heart assembly 102 with a valve and a tubular prosthesis is implanted in the left ventricle 14, the valve may replace the mitral valve 22 and the tubular prosthesis may be positioned upstream of, and adjacent to the aortic valve 20. The prosthetic heart chamber assembly 102 is implanted so that the valve downstream of the tubular prosthesis functions normally.

The prosthetic heart chamber 108 may have a size equal to, or less than, the size of the native heart chamber. When implanted, blood flows into the prosthetic heart chamber 108 through one prosthesis 104, 106 and exits the prosthetic heart chamber 108 through the other prosthesis 104, 106. Thus, the prosthetic heart chamber 108 defines a main lumen 110 that is in fluid communication with the lumen of the first prosthesis 104 and the lumen of the second prosthesis 106.

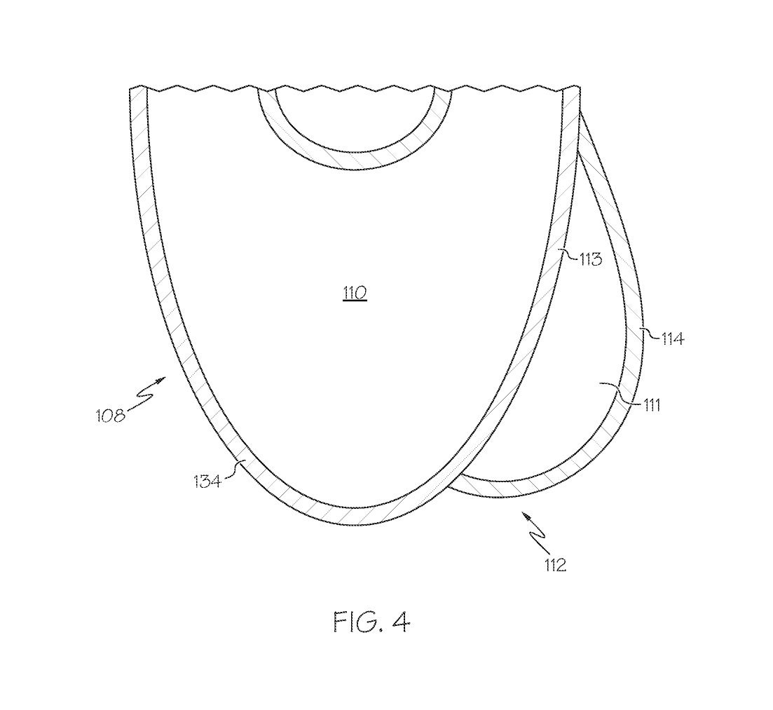

The prosthetic heart chamber 108 may have one or more cooperative mechanism compartments 112 (see e.g. FIGS. 4 and 20). The cooperative mechanism compartment 112 defines a compartment lumen 111 separate from the main lumen 110. The cooperative mechanism compartment 112 may be attached to the wall of the prosthetic heart chamber 108 (see e.g. FIG. 4) or may form a part of the wall of the prosthetic heart chamber 108. The cooperative mechanism compartment 112 may be sized to contain a cooperative mechanism 120, or may be a cooperative mechanism 120. The cooperative mechanism compartment 112 may comprise the same or different material forming the rest of the prosthetic heart chamber 108.

Suitable materials for the prosthetic heart chamber 108 and/or the cooperative mechanism compartment 112 include polyurethane and its copolymers; silicone and its copolymers; ethylene vinyl-acetate; polyethylene terephthalate (PET); thermoplastic elastomers; polyvinyl chloride; polyolefins; cellulosics; polyamides; polyesters; polysulfones; polytetrafluorethylenes; polycarbonates; acrylonitrile butadiene styrene copolymers; acrylics; polycarbonate; poly(glycolide-lactide) copolymer; Tecothane; PEBAX®; polyethylene; polylactic acid; poly(γ-caprolactone); poly(γ-hydroxybutyrate); polydioxanone; poly(γ-ethyl glutamate); polyiminocarbonates; poly(ortho ester); polyanhydrides; polymeric materials described, for example, in U.S. Pat. Nos. 5,650,234 and 5,463,010, herein incorporated in their entirety; and/or blends of these polymers. The material of the prosthetic heart chamber 108 may form a cover that extends over the outer surface of a prosthesis 104, 106 for a portion of the longitudinal length of the prosthesis 104, 106 or for the entire longitudinal length of the prosthesis 104, 106.

Any suitable valve may be used for a prosthesis 104, 106. Valves suitable for the first and second prostheses 104, 106 include, but are not limited to, valves disclosed in the following publications of patents or patent applications, each hereby incorporated by reference herein in their entireties: WO 2005/062980; US 2007/0129788; US 2008/0319526; US 2009/0171456; US 2009/0306768; US 2009/0030512; US 2011/0060405; US 2012/0179239; US 2013/0035758; U.S. Pat. No. 8,070,802; U.S. Pat. No. 7,892,276; U.S. Pat. No. 7,780,722; U.S. Pat. No. 7,670,368; U.S. Pat. No. 7,566,343; U.S. Pat. No. 8,012,198; U.S. Pat. No. 6,685,739; U.S. Pat. No. 7,569,071; U.S. Pat. No. 7,867,274; U.S. Pat. No. 7,776,053; U.S. Pat. No. 7,722,666; U.S. Pat. No. 8,128,681; U.S. Pat. No. 7,416,557; US 2014/0018935; and US 2013/0339866. Additional valves suitable for the first and second valves 104, 106 include valves developed by Boston Scientific Scimed, such as the Lotus Aortic Valve System; valves developed by Edwards, such as the Sapien Transcatheter Heart Valve; valves developed by CardiAQ™ Valve Technologies, such as the CardiAQ prosthesis; valves developed by St. Jude Medical, such as the Trifecta™ valve, the Regent™ aortic valve; and valves developed by Medtronic, such as the Core Valve®.

Any suitable stent, stent-graft, or graft may be used for a prosthesis 104, 106 that is a tubular prosthesis. The tubular prosthesis may have openings in the side wall or may have no openings in the side wall.

One or both of the prostheses 104, 106 may include a means for anchoring the prosthesis 104, 106 (an anchoring means) (not shown). Thus, an “anchoring means” as used herein is a means to minimize or prevent migration of a prosthesis 104, 106. Any suitable anchoring means may be used. Some examples of anchoring means include, but are not limited to, barbs; spikes; anchors; adhesion elements; raised elements or regions of the prosthesis 104, 106; enlarged diameter regions of the prosthesis 104, 106; and means for controlled tissue ingrowth. The anchoring means may extend through a cover positioned on the outer surface of the prosthesis. The anchoring means may be attached to the outer surface of a cover positioned on the outer surface of the prosthesis. The following commonly owned U.S. publications of patents or patent applications, each hereby incorporated by reference herein in their entireties, disclose examples of suitable prosthesis and/or suitable anchoring means for a prosthesis 104, 106 of the prosthetic heart chamber assembly 102: U.S. Pat. No. 8,715,334; 2013/0172983; 2013/0268063; 2014/0277562.

The prostheses 104, 106 may be made from any suitable biocompatible materials including one or more polymers, one or more metals, or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The prostheses 104, 106 may be made of shape memory materials with shape memory effect or superelasticity, such as superelastic Nitinol or spring steel, or may be made of materials which are plastically deformable. In the case of shape memory materials, the prostheses 104, 106 may be provided with a memorized shape and then deformed to a reduced diameter shape. The prostheses 104, 106 may restore itself to its memorized shape upon being heated to a transition temperature and having any restraints removed therefrom. This is known as shape memory effect. Shape memory materials can also be processed to have superelasticity.

The prostheses 104, 106 may be created by methods including cutting or etching a design from a tubular stock, from a flat sheet which is cut or etched and which is subsequently rolled; or from one or more interwoven wires or braids. Any other suitable technique which is known in the art or which is subsequently developed may also be used to manufacture the prostheses 104, 106.

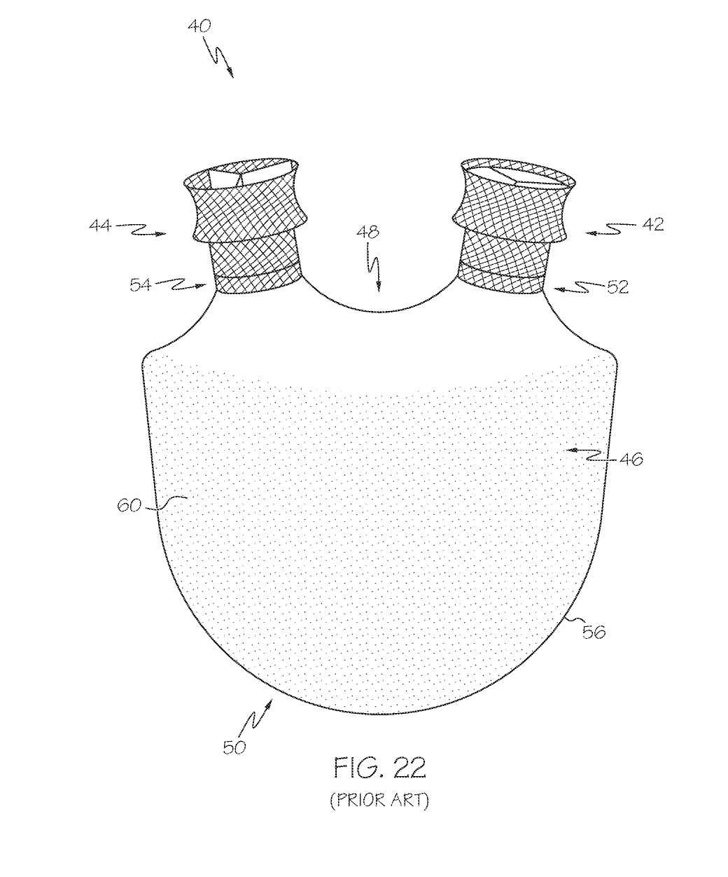

Commonly owned U.S. 2014/0277408 discloses a prosthetic valve assembly 40 suitable for use as a prosthetic heart chamber assembly 102 of a heart system 100 as disclosed herein. The prosthetic valve assembly 40 of U.S. 2014/0277408 comprises a first valve 42, a second valve 44, and a pouch 46 connecting the first and second valves 42, 44 (see FIG. 22). The pouch 46 includes a main compartment 56 with a first span 48 and a second span 50; a first opening 52; and a second opening 54. When the pouch is fully deployed, the pouch may extend to fully occupy the native heart chamber, or may be of a reduced size and/or volume such that the fully deployed pouch occupies only a portion of the native heart chamber. The pouch 46 may also have an adhesive 60 on the outer surface for adherence to the inner surface of the ventricle. U.S. 2014/0277408 discloses that the prosthetic valve assembly 40 may be used for integrated dual valve replacement in the heart, or may be used to treat cardiomyopathy.

The prosthetic heart assembly 102 may form a part of a heart system 100. A heart system 100 as disclosed herein includes a prosthetic heart chamber assembly 102 and a cooperative mechanism 120. The heart system 100 is constructed and arranged to be implanted in a native heart chamber. The heart system 100 may be implanted in either the right ventricle or the left ventricle of a native heart. As discussed below in greater detail, when the heart system 100 is implanted in a heart chamber, the heart system 100 may: partition a dysfunctional heart wall from blood flow or blood contact; assist with the pumping of blood through the heart; prevent further dilation, enlargement, or remodeling of the heart; change the volume of the native heart chamber; change the shape of the native heart chamber; and combinations thereof. Thus, the heart system 100 may be used to treat heart conditions where the function of the heart is impaired, e.g. by damage to the heart muscle (e.g. heart failure or an enlarged heart). For example the heart system 100 may slow or stop the progression of damage to the heart (e.g. slow or stop the progression of heart failure) and/or provide the heart with an opportunity to recuperate from damage. Some benefits of the heart system 100 include reducing surgical ventricular reconstruction operational time and cost, and/or reducing the risk of complications associated with left ventricular assist device placement, including the risk of perioperative bleeding associated with prolonged extracorporeal circulation and hypothermia.

### II. Cooperative Mechanism

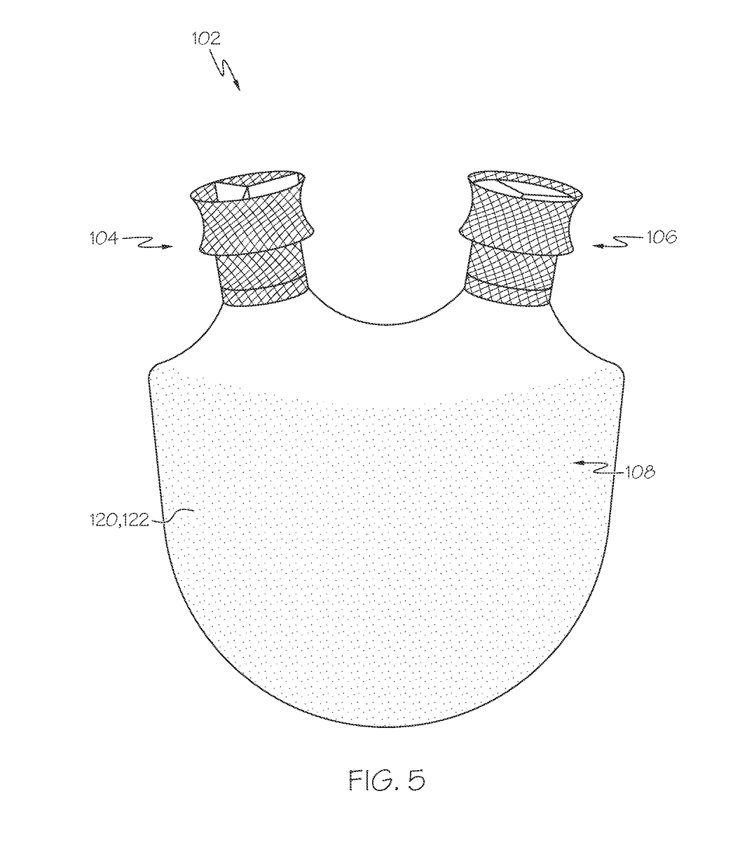

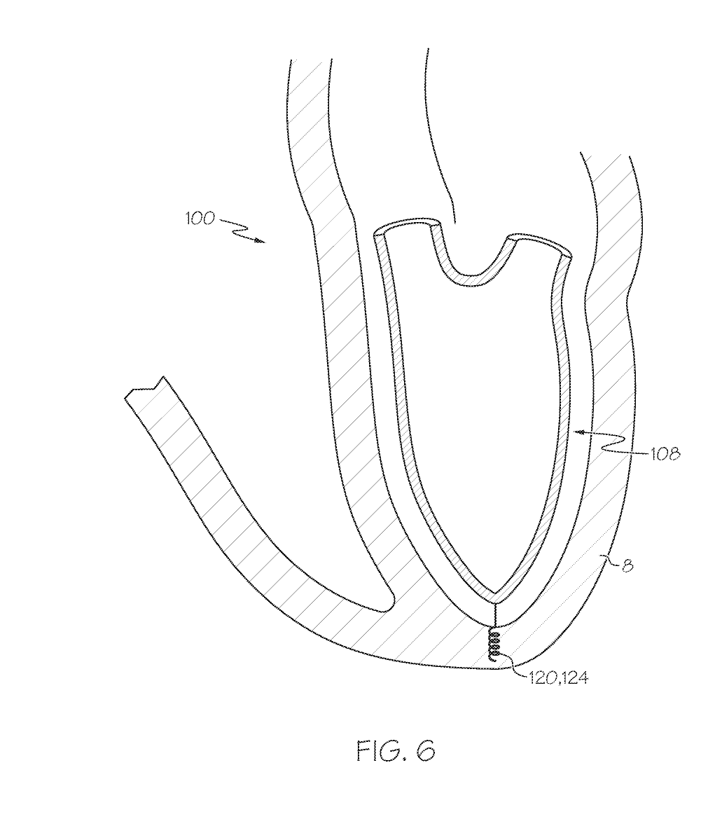

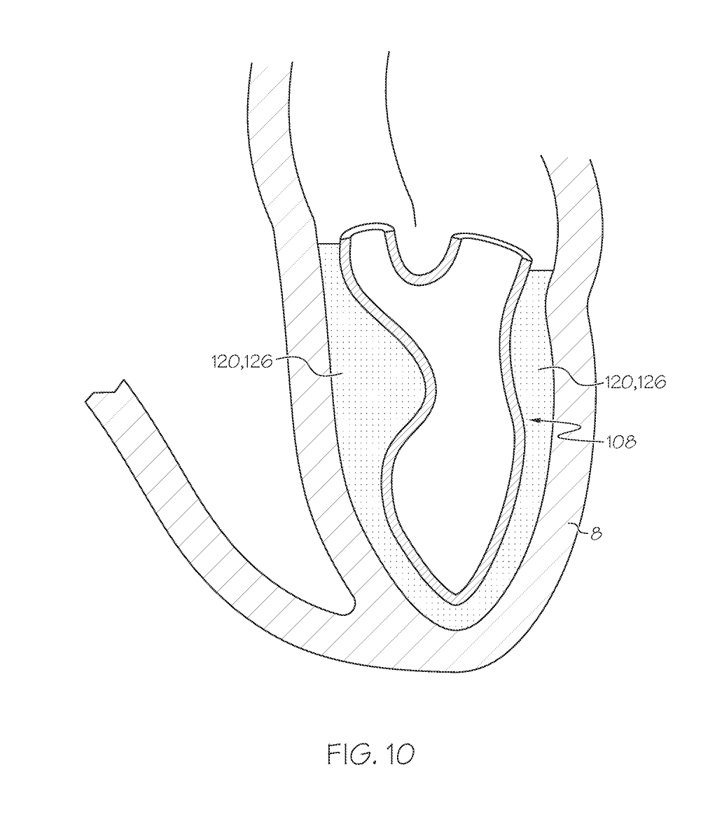

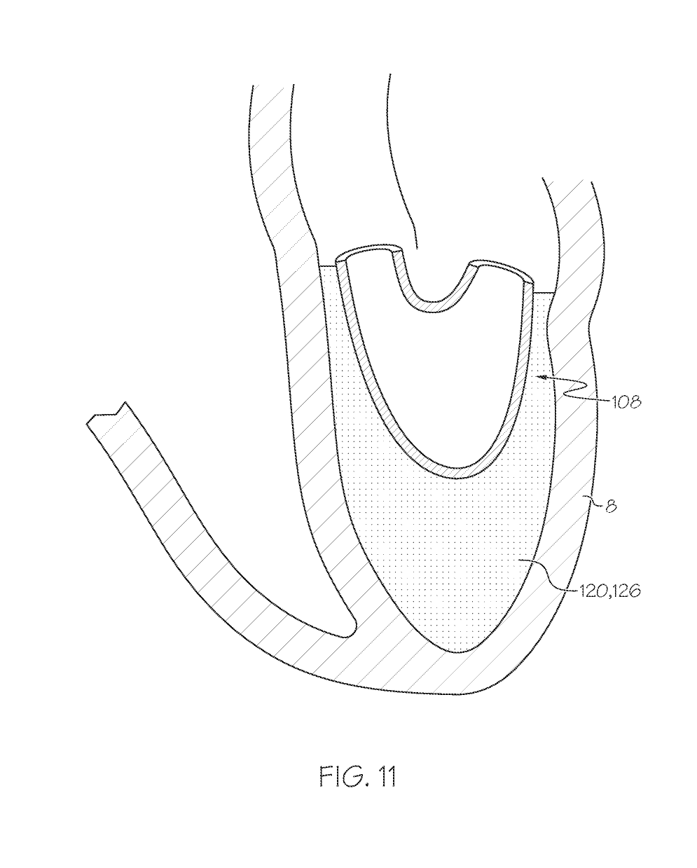

As discussed above, the heart system 100 includes a cooperative mechanism 120 (see e.g. FIGS. 5-21). A “cooperative mechanism” as used herein cooperates with the prosthetic heart chamber 108 to improve the function of the heart and/or provide the native heart with an opportunity to recuperate from damage.

The heart system 100 may have one cooperative mechanism 120 or a plurality of cooperative mechanisms 120. When the heart system 100 is implanted, the cooperative mechanism 120 may be: free floating between the prosthetic heart chamber assembly 102 and the wall 8 of the native heart chamber; secured to the prosthetic heart chamber assembly 102; secured to the wall 8 of the native heart chamber; secured to the prosthetic heart chamber assembly 102 and to the wall 8 of the native heart chamber; positioned within a cooperative mechanism compartment 112; or form a part of the prosthetic heart chamber 108.

### II.A. Examples of Cooperative Mechanisms

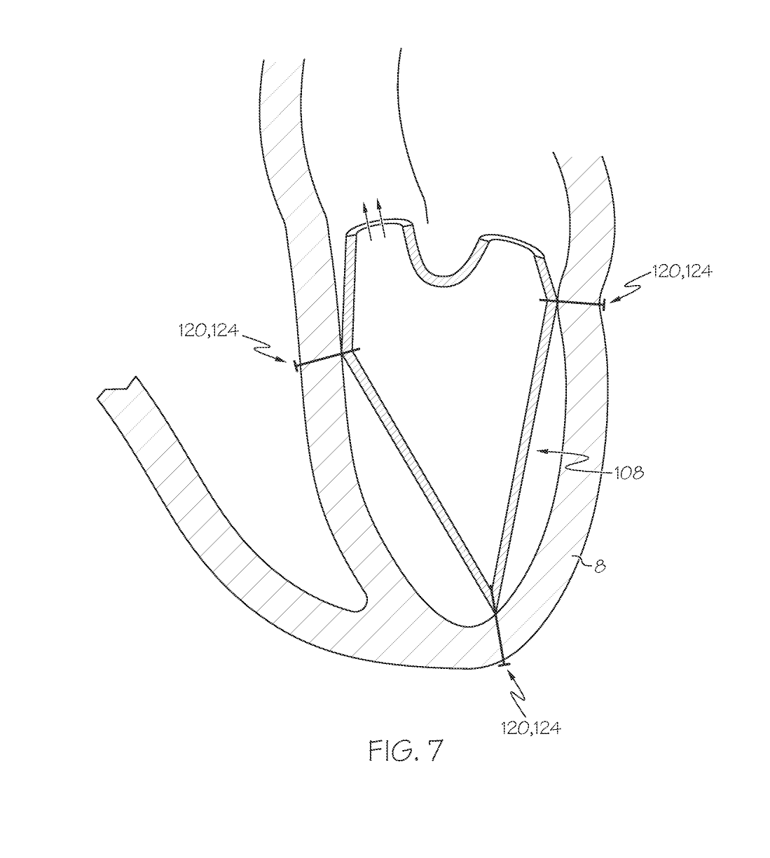

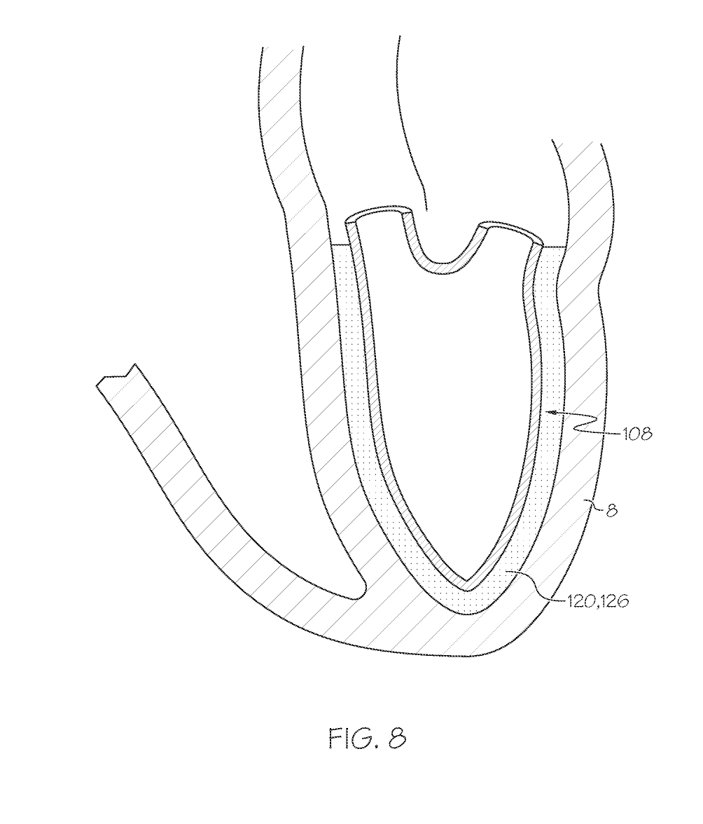

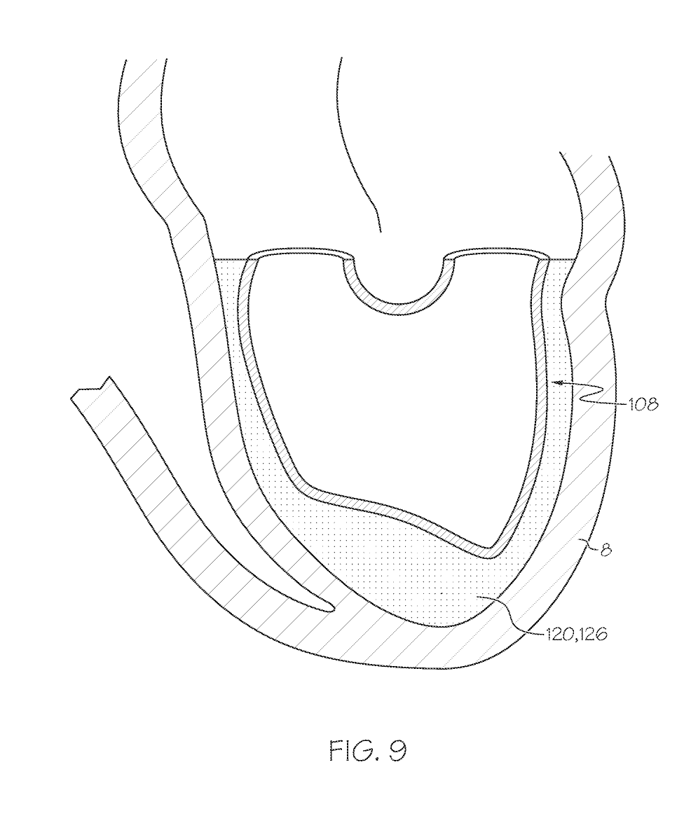

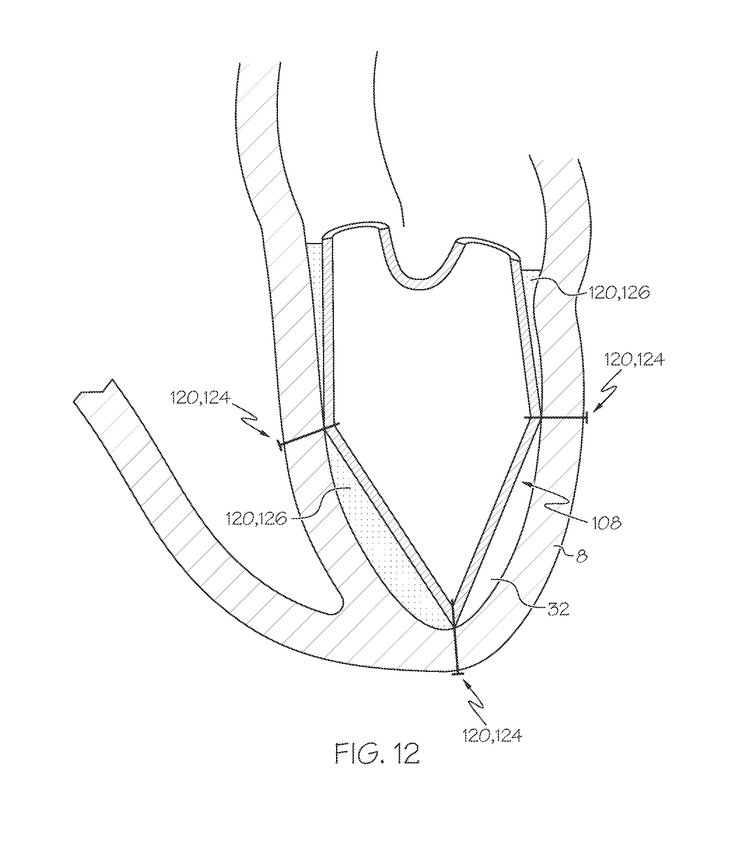

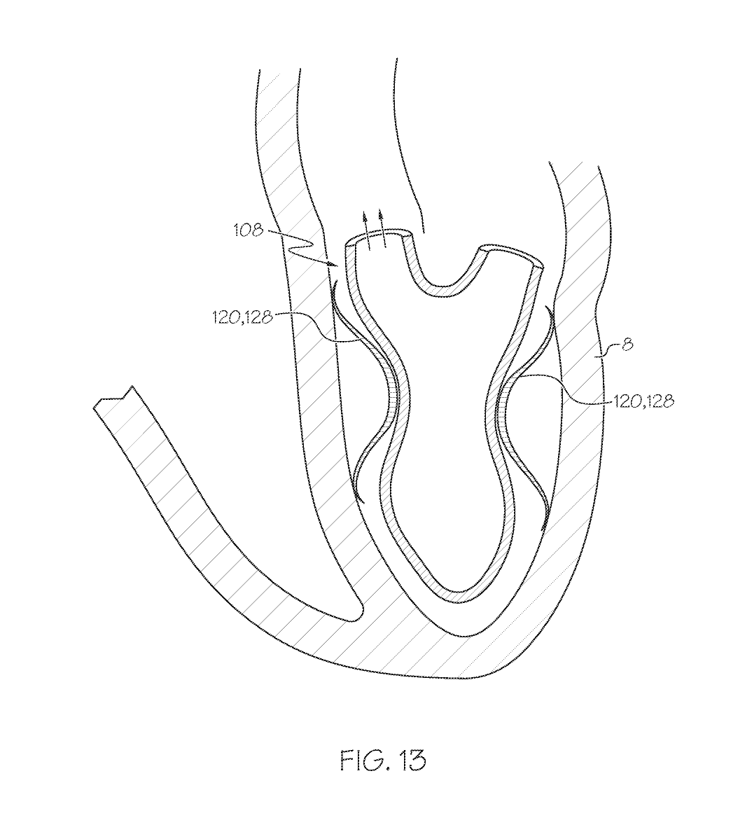

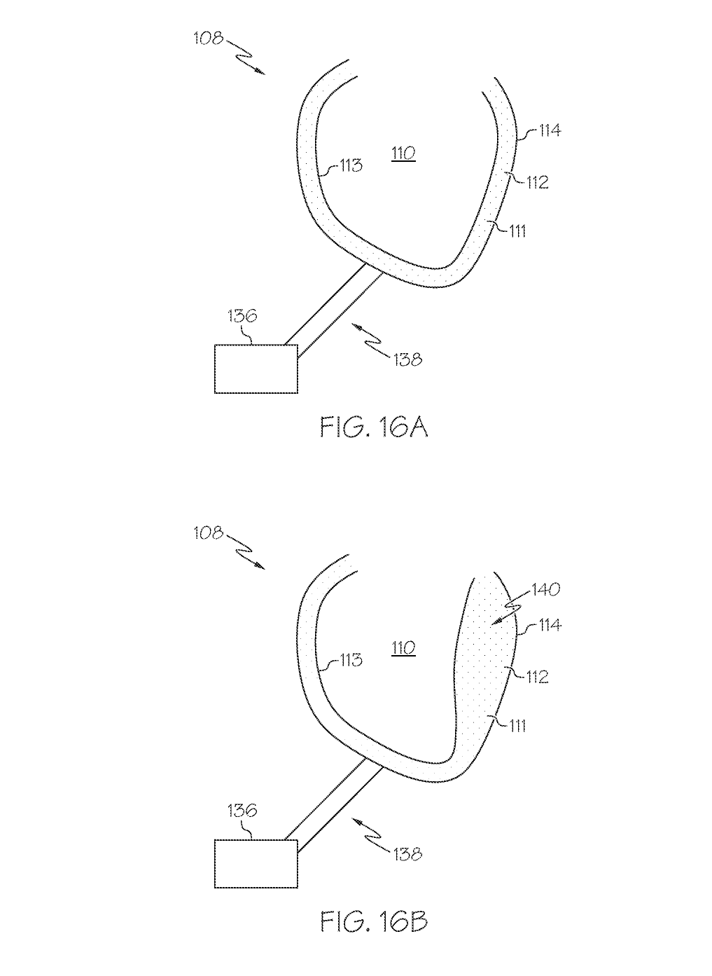

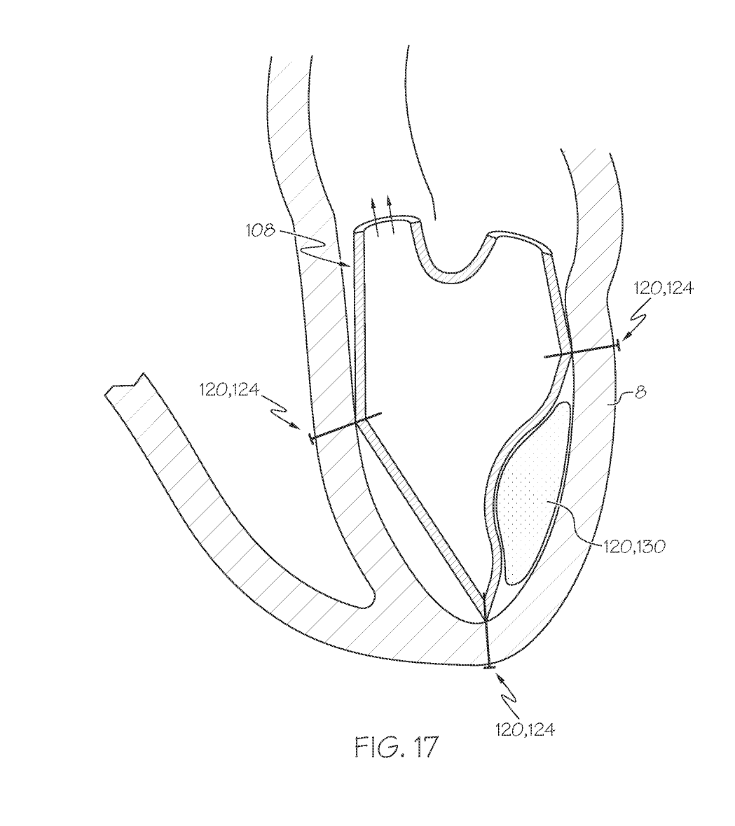

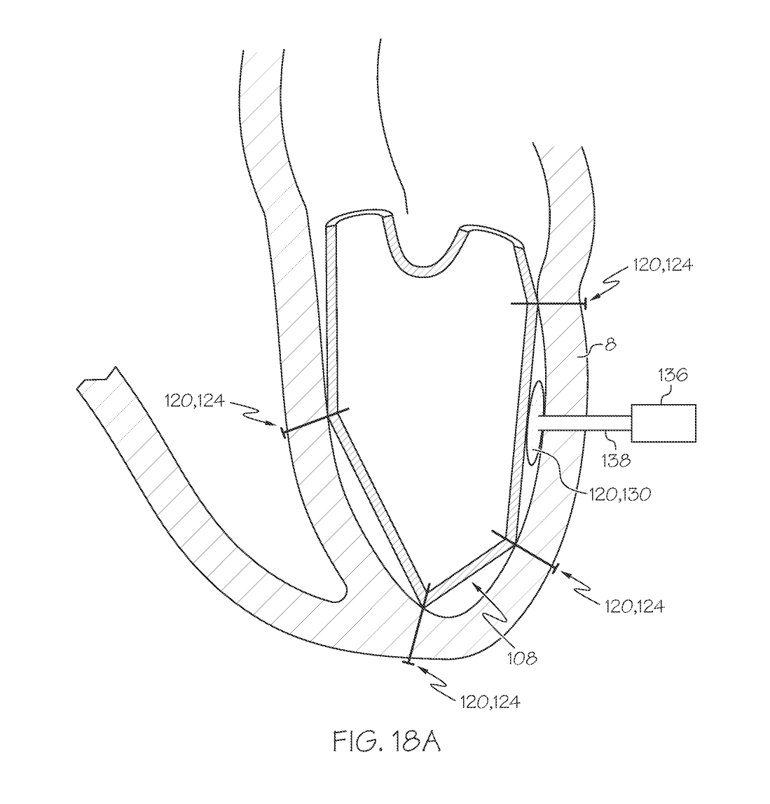

The cooperative mechanism 120 may be an adhesive 122 (see e.g. FIG. 5); an anchor 124 (see e.g. FIGS. 6-7); a filler material 126 (see e.g. FIGS. 8-12); a shape memory element 128 (see e.g. FIG. 13-15); a pump device 136 (FIGS. 16, 18 and 20); a cooperative mechanism compartment 112 (see e.g. FIGS. 16 and 20); an expandable member 130 (see e.g. FIGS. 17-20); a material 134 attached to, or forming a part of, the wall of the prosthetic heart chamber 108 (e.g. FIGS. 3-4, 12, and 21); and combinations thereof (see e.g. FIGS. 12, 14-15, and 18-20).

### II.A.1. Adhesive

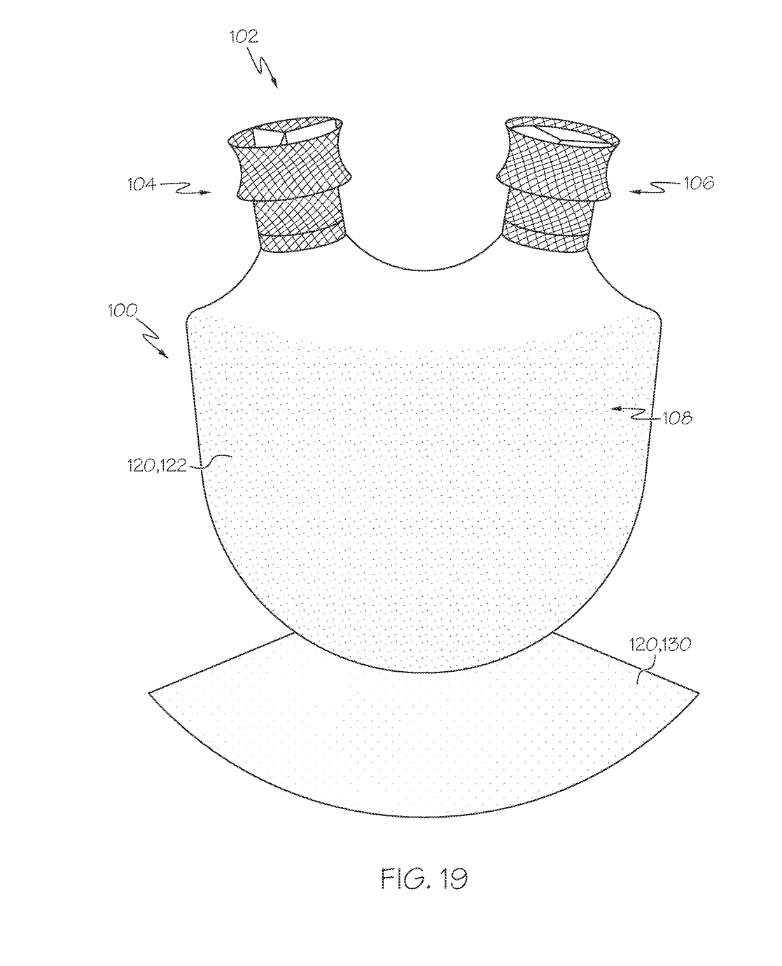

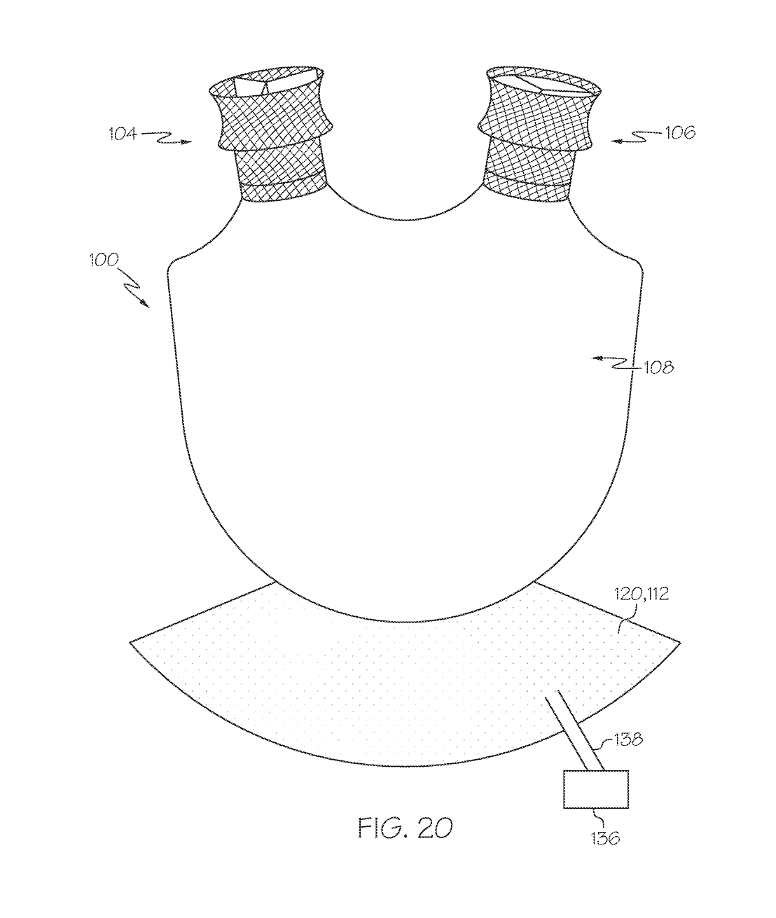

As discussed above, the cooperative mechanism 120 may be an adhesive 122 (see e.g. FIGS. 5 and 19). An adhesive 122 may cover the entire outer surface of the prosthetic heart chamber 108 or only a portion of the outer surface of the prosthetic heart chamber 108. The adhesive 122 may attach the prosthetic heart chamber 108 to the wall 8 of a native heart chamber, or may attach another cooperative mechanism 120 to the prosthetic heart chamber 108. In these ways, the adhesive 122 cooperates with the other structures of the heart system 100 to improve the function of the heart and/or provide the native heart with an opportunity to recuperate from damage.

Adhesives suitable for adhesive 122 include natural polymeric materials, as well as synthetic materials, and synthetic materials formed from biological monomers such as sugars. Bioadhesives can also be obtained from the secretions of microbes or by marine mollusks and crustaceans. Bioadhesives are designed to adhere to biological tissue. In at least one embodiment, the adhesive activity of the adhesive layer is controlled through compound design such that an exposure time is required for tracking the device to the heart before the adhesive is ready to bond to the ventricle or other lumen wall.

Examples of bioadhesives include, but are not limited to, amino adhesives, adhesive surface proteins (MSCRAMMS), adhesively modified biodegradable polymers such as Fatty Ester Modified PLA/PLGA, polymer materials, minigel particles, each discussed in detail below, as well as mixtures thereof.

Suitably, the bioadhesive is dissolved in a solvent or cosolvent blend prior to application. Suitable solvents include, but are not limited to, alcohols including methanol, ethanol and isopropanol, and water.

The following examples of bioadhesives are intended for illustrative purposes only, and not as a limitation on the scope of the present disclosure.

1. Amino Acids: Amino acids can be utilized to facilitate adhesion to the lesion site. Zwitterionic amino acids can be employed as a layer or as a component within prosthetic chamber layer. The zwitterionic amino acid can be oriented so that the hydrophobic side of the zwitterionic amino acid selectively facilitates adhesion to the lipophilic heart wall. One example of a useful compound is amino acid 3,4-L-dihydroxyphenylalanine (DOPA), a tyrosine derivative found in high concentrations in the “glue” proteins of mussels.

2. Adhesive Surface Proteins: Protein adhesions called MSCRAMMs (microbial surface components recognizing adhesive matrix molecules) can also be employed as a bioadhesive in the second coating composition. MSCRAMMS are naturally produced by pathogens to initiate adhesion to the host extra cellular matrix to initiate infection. These adhesive surface proteins can be isolated or synthesized and utilized as a separate layer to facilitate adhesion the lesion site.

3. Adhesively Modified Biodegradable Polymers: One example of an adhesively modified biodegradable polymer is a DOPA (L-3,4-dihydroxyphenylalanine) modified PLA (polylactic acid), or PLGA poly(lactide-co-glycolide). In this embodiment, examples of suitable adhesive moieties include, but are not limited to, monopalmitate, monostearin, glycerol, and dilaurin or iso-stearyl alcohol.

4. Polymer Materials: Proteins such as gelatin and carbohydrates such as starch may also be employed. Polysaccharides such as sorbitol, sucrose, xylitol, anionic hydrated polysaccharides such as gellan, curdlan, XM-6, and xanthan may also be employed as a bioadhesive. Others include derivatives of natural compositions such as algenic acid, hydrated gels and the like, and also biocompatable polymers and oligomers such as dextrans, dextranes and dextrins, hydrogels including, but not limited to, polyethylene glycol (PEG), polyethylene glycol/dextran aldehyde, polyethylene oxide, polypropyline oxide, polyvinylpyrrolidine, polyvinyl acetate, polyhydroxyethyl methacrylate and polyvinyl alcohol, as well as derivatives thereof may also be employed herein. See for example U.S. Pat. No. 6,391,033 and Aldehyde-Amine Chemistry Enables Modulated Biosealants with Tissue-Specific Adhesion by Artzi et al. (Advanced Materials Vol. 21, Issue 32-33, pages 3399-3403) the entire content of each is incorporated by reference herein.

5. Minigel Particles: Another bioadhesive is poly(NIPAM) (poly(N-isopropylacrylamide) minigel particles. This polymer has the property of being in a liquid state at room temperature and an adhesive at body temperature., see “Preparation and Swelling Properties of Poly (NIPAM) “Minigel” Particles Prepared by Inverse Suspension Polymerization”, Dowding, John et al., Journal of Colloid and Interface Science 221, 268-272 (2000), available online at http:/www.idealibrary.com, the entire content of which is incorporated by reference herein.

For better retention of a polymer adhesive on the surface of the prosthetic heart chamber 108, several techniques may be employed. Suitably, the minigel particles are crosslinked or mixed with a higher molecular weight polymer to allow enough time for retention of the minigel to the medical device during delivery, or uncrosslinked minigel particles can be employed in a crosslinked polymer network.

### II.A.2. Anchor

As discussed above, the cooperative mechanism 120 may be an anchor 124 (see e.g. FIGS. 6-7, 12, 14-15, 17-18). Although the first and second prostheses 104, 106 may be considered to function as “anchors,” as used in this disclosure, an anchor 124 is not a prosthesis 104, 106. Also, an anchor 124 is not an anchoring means of a prosthesis 104, 106, but instead the anchor 124 is an additional structure attached to the prosthetic heart chamber 108 and constructed and arranged to penetrate into the wall 8 of the native heart chamber.

The heart system 100 may include one anchor 124 (see e.g. FIG. 6), or a plurality of anchors 124 (see e.g. FIG. 7) to attach the prosthetic heart chamber 108 to the wall 8 of the native heart chamber. In these ways, the anchor 124 cooperates with the other structures of the heart system 100 to improve the function of the heart and/or provide the native heart with an opportunity to recuperate from damage.

The anchor 124 may have any suitable configuration for attaching the prosthetic heart chamber 108 to the wall 8 of the native heart chamber. For example, the anchor 124 may have an end region that spirals (see e.g. FIG. 6), the shaft of the anchor may have barbs; or the anchor may have a shaft with elements that spread radially outwards from the shaft once the elements have reached the outer surface of the native heart chamber. The distal end of the anchor 124 may be positioned inside the wall 8 of the native heart chamber (see e.g. FIG. 6) or outside the wall 8 of the native heart chamber (e.g. anchor has a length greater than the thickness of the wall of the native heart chamber).

### II.A.3. Filler Material

As discussed above, the cooperative mechanism 120 may be a filler material 126 (see e.g. FIGS. 8-12). When the heart system 100 is implanted, filler material 126 may surround the entire prosthetic heart chamber 108 (see e.g. FIG. 8) or may surround only a portion of the prosthetic heart chamber 108 (see e.g. FIG. 12).

The filler material 126 may be positioned in a cooperative mechanism compartment 112 of the prosthetic heart chamber 108 or be free floating between the prosthetic heart chamber 108 and the wall 8 of the native heart chamber. Where the filler material 126 is free floating, the first and second prostheses 104, 106 of the prosthetic heart chamber assembly 102 prevent the filler material 126 from moving out of the native heart chamber. For example, expansion of the first and second prostheses 104, 106 provides a seal between the prosthetic heart chamber assembly 102 and the native heart chamber. Adhesive 122 adjacent to the first and second prostheses 104, 106 may additionally be used to provide a seal between the prosthetic heart chamber assembly 102 and the native heart chamber and to maintain the filler material 126 within the native heart chamber.

The filler material 126 may be attached to the prosthetic heart chamber 108. The filler material 126 may attach to the prosthetic heart chamber 108 by itself, or the filler material 126 may be attached to the prosthetic heart chamber 108 by an adhesive 122.

Suitable materials for the filler material 126 include but are not limited to, foam forming materials; thixotropic materials; scaffolding materials; liquids; and combinations thereof. As used in this disclosure, a “thixotropic material” is a material that is thick or viscous under static conditions, and thin or less viscous when shaken, agitated, or otherwise stressed.

Examples of suitable foams include: biodegradable biomedical foams; bioactive glass foams, such as those used for tissue engineering applications; polyurethane-based shape memory polymer foams; CHEM (cold hibernated elastic memory) foams; bioactive glass and glass-ceramic foam scaffolds such as those used for bone tissue restoration; composite biomedical foams, such as those used for engineering bone tissue; injectable biomedical foams, such as those used for bone regeneration; polylactic acid (PLA) biomedical foams, such as those used for tissue engineering; porous hydrogel biomedical foam, such as those used for scaffolds for tissue repair; and titanium biomedical foams, such as those used for osseointegration. Commonly occurring examples of foam forming polymers include polyurethane, Neoprene, Expanded Polystyrene (EPS). There is a wide range of different biomaterial materials may be used for biodegradable foams, including, but not limited to, ethylene vinyl alcohol; polyvinyl alcohol; polycaprolactone; polylactic acid; and starch.

Examples of suitable thixotropic materials include poly saccharide based hydrogels that demonstrate thixotropic behaviour; and synovial fluid, which is a thixotropic fluid. Connective tissue may also be used. Connective tissue contains ‘ground substance’ (extrafibrillar matrix), which is a thixotropic gel-like substance surrounding the cells, and contains a mixture of extracellular matrix components (water, glycosaminoglycans (most notably hyaluronan), proteoglycans, and glycoproteins.

Examples of suitable liquids include saline, water, and blood.

Another example of a suitable scaffolding material is hyaluronan or hyaluronic acid.

### II.A.4. Shape Memory Element

As discussed above, the cooperative mechanism 120 may be a shape memory element 128 (see e.g. FIGS. 13-15). The shape memory element 128 may comprise a shape memory alloy with superelasticity (e.g. Nitinol); or a shape memory polymer (e.g. styrene transbutadiene-styrene triblock co-polymer). A shape memory alloy with superelasticity undergoes a phase transformation due to stress, otherwise referred to as a stress-induced martensitic transformation. Shape memory polymers have a high capacity for elastic deformation (up to 200% in most cases).

A shape memory element 128 comprising a shape memory alloy may also have a shape memory effect for loading onto a delivery system. For example, a cooperative mechanism 120 with a shape memory effect may be cooled down to facilitate loading onto a delivery device.

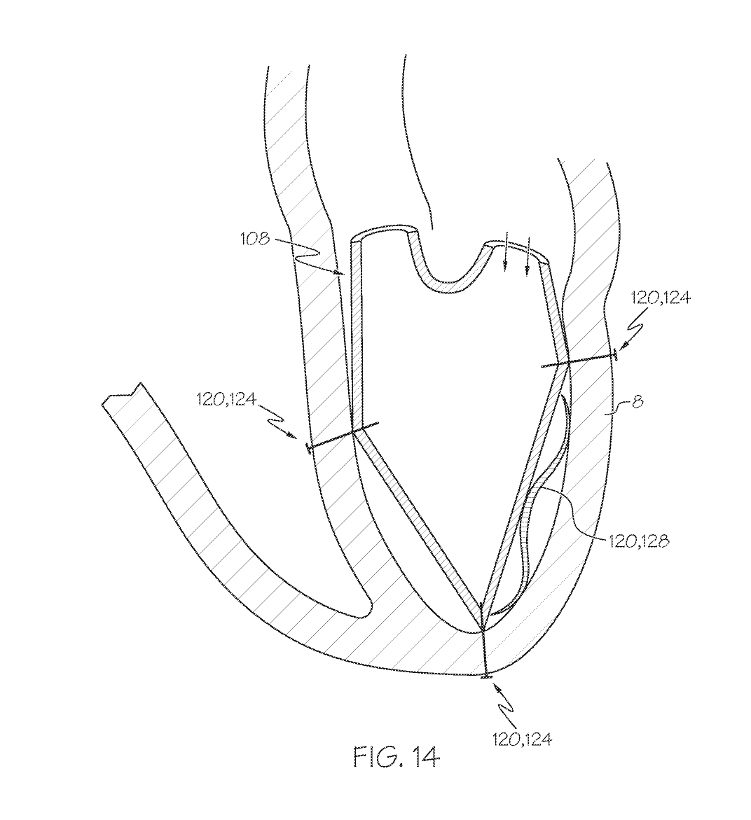

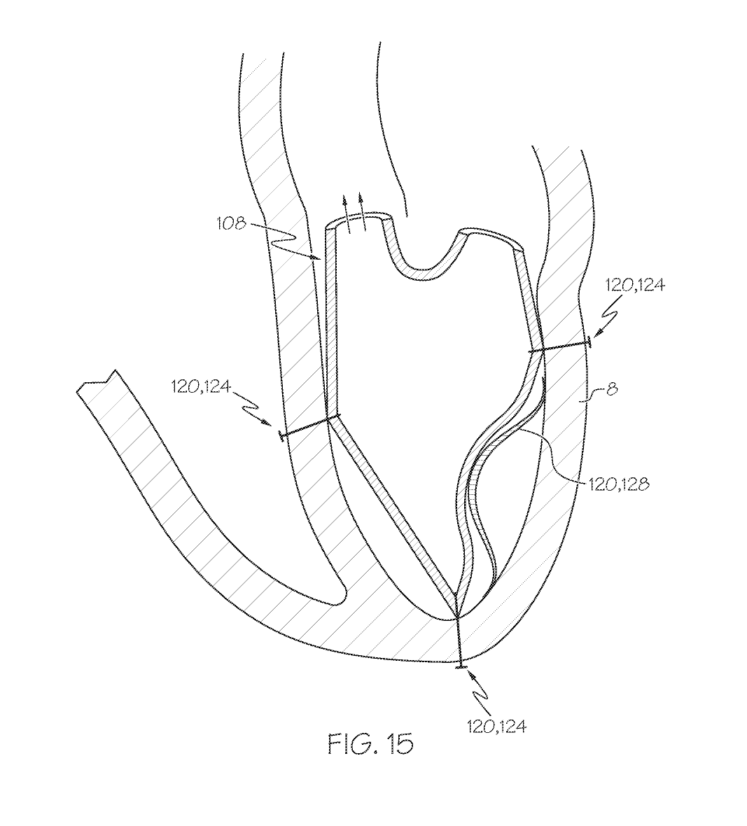

The shape memory element 128 has a first shape during diastole (e.g. FIG. 14) and a second shape during systole (see e.g. FIG. 15). The shape of the shape memory element 128 during systole is an undeformed shape. The shape memory element 128 can have any atraumatic shape so that it does not puncture the chamber wall. At least a portion of the shape memory element 128 in the undeformed configuration may press against the prosthetic heart chamber 108 so that the size of the main lumen 110 is reduced.

The shape memory element 128 may be elongated. The shape memory element 128 may have a round shape; or a rectangular or ribbon-like shape. The shape memory element may have edges that are rounded or blunt. The shape memory element 128 may have ends that are rounded or blunt. The shape memory element 128 may have end regions that are curled or curved (see e.g. FIGS. 13-15). As used in this disclosure, an “end” is the last part or extremity of an element, while an “end region” is a region adjacent to, and includes, the “end.” The shape memory element 128 may be a wire with atraumatic ends.

The shape memory element 128 may be free floating between the prosthetic heart chamber assembly 102 and the wall 8 of the native heart chamber (see e.g. FIG. 13), or positioned within a cooperative mechanism compartment 112 of the prosthetic heart chamber 108 of the prosthetic heart chamber assembly 102. The shape memory element 128 may be secured to the prosthetic heart chamber assembly 102; or form a part of the prosthetic heart chamber 108.

### II.A.5. Pump Device

As discussed above, the cooperative mechanism 120 may be a pump device 136 (see e.g. FIGS. 16, 18, and 20). The pump device 136 is constructed and arranged to modulate the size of the main lumen 110 to aid movement of blood through the heart. The pump device 136 may include any suitable means to pump expansion media into and out of the expandable member 130.

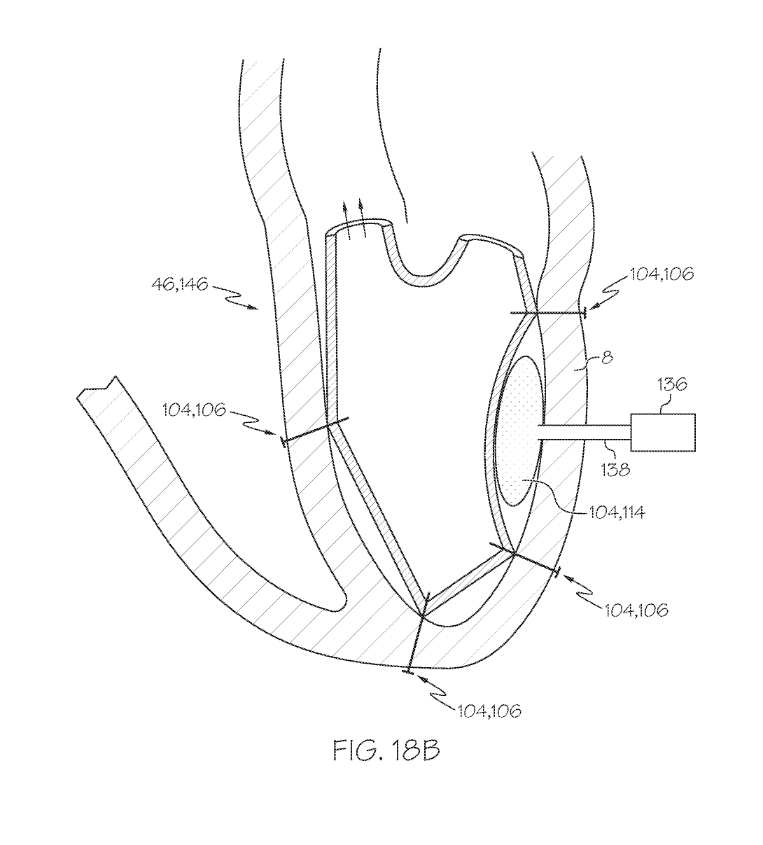

The pump device 136 may be attached to, and in fluid communication with a cooperative mechanism 120. The cooperative mechanism may be an expandable member 130 (see e.g. FIG. 18), or a cooperative mechanism compartment 112 of the prosthetic heart chamber 108 (see e.g. FIGS. 16 and 20).

The pump device 136 may be connected to the patient's heart in the same way that a ventricular assist device is attached to a patient's heart. For example an elongated hollow tube 138 may be in fluid communication with a cooperative mechanism 120 and the pump device 136. The elongated hollow tube 138 may have a first end connected to the cooperative mechanism 120, a portion of the elongated hollow tube 138 extending through the chamber wall, and a second end of the elongated hollow tube 138 attached to the pump device 136. The elongated hollow tube 138 may also extend through a small hole of the abdomen so that the pump device 136 may be carried external to the patient's body, like the control unit of a ventricular assist device.

### II.A.6. Cooperative Mechanism Compartment

As discussed above, the cooperative mechanism 120 may be the cooperative mechanism compartment 112 of the prosthetic heart chamber 108 (see e.g. FIGS. 16 and 20). The cooperative mechanism compartment 112 may be constructed and arranged to modulate the size of the main lumen 110 to aid movement of blood through the prosthetic heart chamber 108. The outer wall 114 of the cooperative mechanism compartment 112 may form the outer surface of the prosthetic heart chamber 108 and the inner wall 113 of the cooperative mechanism compartment 112 main define a portion of the main lumen 110.

The cooperative mechanism compartment 112 may be constructed and arranged to increase and decrease in size. Thus, the cooperative mechanism compartment 112 can be described as an expandable cooperative mechanism compartment. For example, during systole the size of the cooperative mechanism compartment 112 increases and during diastole the size of the cooperative mechanism compartment 112 decreases. Thus the size of the cooperative mechanism compartment 112 during systole is greater than the size of the cooperative mechanism compartment 112 during diastole. During systole, the cooperative mechanism compartment 112 may extend inward into the main lumen 110 (see e.g. FIG. 16B), or outward towards the heart chamber wall (see e.g. FIG. 20). In either case the cooperative mechanism compartment 112 decreases the size of the main lumen 110 to eject blood from the prosthetic heart chamber 108. Thus, the main lumen 110 of the prosthetic heart chamber 108 has a smaller size during systole than during diastole.

The size of the cooperative mechanism compartment 112 may be modulated by the pump device 136 (see e.g. FIGS. 16A-B). When the heart system 100 is implanted, the pump device 136 pumps a volume of expansion media into the cooperative mechanism compartment 112 during systole, and during diastole the pump device 136 withdraws a volume of expansion media from the cooperative mechanism compartment 112.

Any suitable expansion/inflation media 140 may be used to expand the cooperative mechanism compartment 112. The expansion media may be a liquid or a gas.

The inner wall 113 of the cooperative mechanism compartment 112 may comprise an elastic material or a non-elastic material. An inner wall 113 comprising an elastic material will expand inward into the main lumen 110 when expansion media is pumped into the cooperative mechanism compartment 112 (see e.g. FIG. 16B). Conversely, an inner wall 113 comprises a non-elastic material will not expand inward into the main lumen 110 when expansion media is pumped into the cooperative mechanism compartment

The outer wall 114 of the cooperative mechanism compartment 112 may comprise an elastic material or a non-elastic material. An outer wall 114 comprising an elastic material will expand outward when expansion media is pumped into the cooperative mechanism compartment 112 (see e.g. FIG. 20). Conversely, an outer wall 114 comprising a non-elastic material will not expand outward when expansion media is pumped into the cooperative mechanism compartment (see e.g. FIG. 16B).

Only one of the inner and outer walls 113, 114 of the cooperative mechanism compartment 112 may comprise an elastic material or both the inner and outer walls 113, 114 of the cooperative mechanism compartment 112 may comprise an elastic material. Where both the inner and outer walls 113, 114 are elastic, the inner wall 113 may extend inwards into the main lumen 110 and the outer wall 114 may extend outwards towards the heart chamber wall when expansion media is pumped into the cooperative mechanism compartment 112.

### II.A.7. Expandable Member

As discussed above, the cooperative mechanism 120 may be an expandable member 130 (see e.g. FIGS. 17-18). An “expandable member” as used herein defines an enclosed lumen and is expandable/retractable between a first size (see e.g. FIG. 18A) and a second size (see e.g. FIG. 18B).

The expandable member 130 may be free floating next to the prosthetic heart chamber 108 (see e.g. FIG. 17); may be attached to the prosthetic heart chamber 108; or may form a part of the prosthetic heart chamber 108. An expandable member 130 as shown in FIG. 19 may be attached to the prosthetic heart chamber 108 or may form a part of the prosthetic heart chamber 108.

The expandable member 130 may be inflatable or expandable. Examples of expandable members 130 include a balloon and a cooperative mechanism compartment 112. A cooperative mechanism compartment 112 constructed and arranged to expand and retract is discussed above in II.A.6. The balloon may be free floating next to the prosthetic heart chamber 108 or may be attached to the prosthetic heart chamber 108.

The expandable member 130 may be inflatable from a delivery size to a deployed size. The deployed size of the expandable member 130 may be modified after the heart system 100 is implanted. For example, expansion/inflation media may be added to, or extracted from, the expandable member to increase or decrease the deployed size of the expandable member after implantation of the heart system 100. Any suitable inflation media may be used. The inflation media may be a liquid or a gas.

The expandable member 130 may have the same deployed size when the heart is in systole and when the heart is in diastole. In other words, the deployed size of the expandable member 130 does not change as the heart pumps blood.

The expandable member 130 may have a first deployed size when the heart is in systole and a second, smaller, deployed size when the heart is in diastole (see e.g. FIGS. 18A-B). Modifying the deployed size of the expandable member 130 may aid the heart in pumping blood. To modify the deployed size after implantation, the expandable member 130 is in fluid communication with a pump device 136. Similar to the modulation of the size of the cooperative mechanism compartment 112 by the pump device 136, the pump device 136 is connected to the heart system 100 so that during systole the pump device 136 pumps a volume of inflation media into the expandable member 130 and during diastole the pump device 136 withdraws a volume of inflation media from the expandable member 130. By increasing the deployment size of the expandable member 130 during systole, the expandable member 130 presses against the prosthetic heart chamber 108 and aids in the ejection of blood from the prosthetic heart chamber 108.

The pump device 136 may be connected to the expandable member 130 in a similar way it is connected to the cooperative mechanism compartment 112. For example an elongated hollow tube 138 may be have a first end connected to the expandable member 130, a portion of the elongated hollow tube 138 may extend through the chamber wall, and a second end of the elongated hollow tube 138 may be attached to the pump device 136. The elongated hollow tube 138 may also extend through a small hole of the abdomen so that the pump device 136 may be carried external to the patient's body, like the control unit of a ventricular assist device. The pump device 136 may include any suitable means to pump inflation media into and out of the expandable member 130.

### II.A.8. Material Attached to, or Forming a Part of, The Wall of the Prosthetic Heart Chamber

As discussed above, the cooperative mechanism 120 may be material 134 attached to, or forming a part of, the wall of the prosthetic heart chamber 108 (see e.g. FIGS. 3-4, 12, and 21). The material 134 is constructed and arranged to return to an original shape from a deformed enlarged shape thereby mechanically facilitating the prosthetic heart chamber 108 in the function of systole. For example, the material 134 may be an elastic material that returns to an original shape and size after expansion and release of stress forces; or an electroactive polymer constructed and arranged to change size and/or shape when stimulated by an electric field (i.e. an electric electroactive polymer).

Examples of suitable elastic materials include rubber, elastomeric polymers, latex, polyurethanes, silicone, and combinations thereof.

The prosthetic heart chamber 108 may have an elastic region and a non-elastic region. A prosthetic heart chamber 108 with elastic and non-elastic regions may be used to partition a section of dysfunctional heart wall from blood flow or blood contact. For example, as discussed in greater detail in section IV.A., an elastic region may be held taut by anchors to partition a section of dysfunctional heart wall.

The elastic region may comprise an elastic material; a shape memory material; and combinations thereof. The shape memory material may be a shape memory alloy such as Nitinol. The shape memory material may be a braid or a knit. An elastic region comprising a shape memory material may be constructed and arranged to be biased to a contracted configuration that assists with blood ejection.

The non-elastic region(s) may comprise non-compliant materials. Examples of suitable non-compliant materials, include, but are not limited to, polyether block amides (e.g. Pebax®); polyethylene terephthalate (PET); poly ether ketone (PEEK); and combinations thereof.

An area of the prosthetic heart chamber 108 may be made into a non-compliant region by adding reinforcement. For example, reinforcing fibers may be attached to, or embedded in, an area of the prosthetic heart chamber 108 to form a non-compliant region(s). The reinforcement may comprise fibers. The reinforcement may comprise a para-aramid synthetic fiber (e.g. Kevlar®); a metallic material; and combinations thereof.

A prosthetic heart chamber 108 comprising an electric electroactive polymer has a first shape when the heart is in diastole and a second, different, shape when the heart is in systole. The second shape of the electroactive polymer is constructed and arranged to aid in the ejection of blood from the prosthetic heart chamber. Thus, the second shape of the electroactive polymer may be constructed and arranged to reduce the size of the main lumen 110 of the prosthetic heart chamber 108.

The electroactive polymer may be in one or more sections. The sections can have any size (length, width). The section(s) of electroactive polymer may be attached to the wall of the prosthetic heart chamber 108, or may form a part of the wall of the prosthetic heart chamber 108.

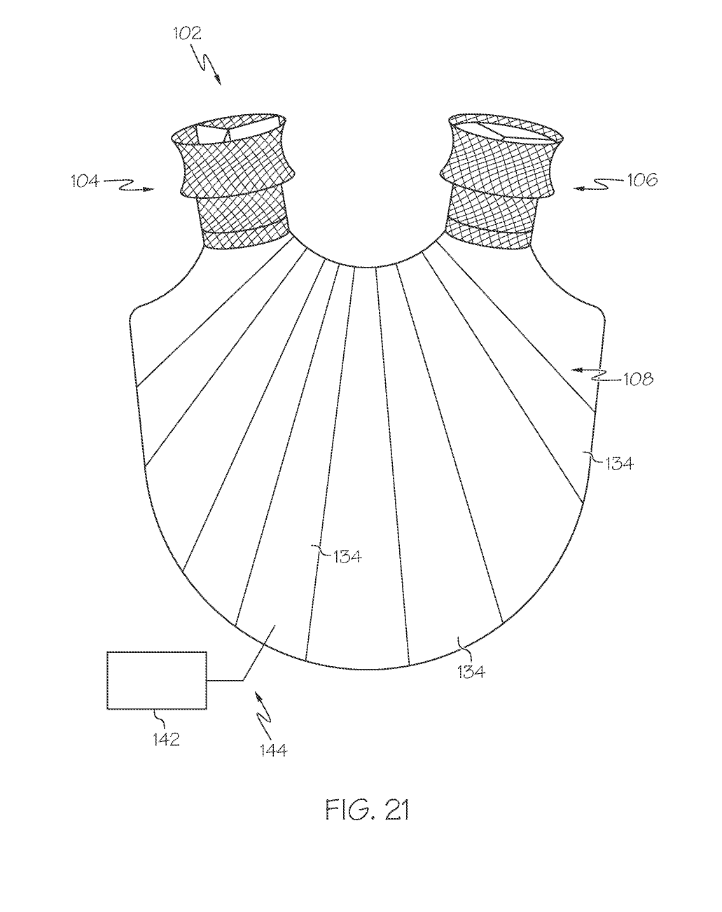

The section(s) of electroactive polymer may have any suitable configuration that promotes blood ejection from the prosthetic heart chamber 108. For example, the electroactive polymer may form the entire outer surface of the prosthetic heart chamber 108. The electroactive polymer may form only a portion of the outer surface of the prosthetic heart chamber 108. For example, the sections of electroactive polymer may be bands of electroactive polymer 134 (see e.g. FIG. 21). The bands of electroactive polymer may be attached so that the bands extend from the top of the prosthetic heart chamber 108 (e.g. adjacent to the first and second prostheses 104, 106) to the bottom of the prosthetic heart chamber 108; may be attached so that the bands extend circumferentially around the prosthetic heart chamber 108; or may be attached so that the bands of electroactive polymer extend diagonally around the prosthetic heart chamber 108.

Where there is a plurality of sections of electroactive polymer, the sections of electroactive polymer may be constructed and arranged to be actuated in sequence. For example, section(s) of electroactive polymer attached to the bottom portion of the prosthetic heart chamber 108 may be actuated before the section(s) of electroactive polymer attached to the top portion of the prosthetic heart chamber are actuated. This arrangement may promote flow of blood from the bottom of the heart upwards through the aorta.

As discussed above, an electric electroactive polymer changes size and/or shape when stimulated by an electric field. The electric field may be provided from an electric field generating device 142 in association with the prosthetic heart chamber assembly 102 (see e.g. FIG. 21). Any suitable device may be used to generate an electric field to stimulate the electroactive polymer. For example, the electric field may be generated by a battery of the electric field generating device 142. Thus, the electric field generating device 142 may be described as being in electrical communication 144 with the prosthetic heart chamber assembly 102.

The electric field generating device 142 may be directly or indirectly attached to the prosthetic heart chamber assembly 102. The electric field generating device 142 may be implanted into a patient's body or carried outside of the patient's body. For example, the electric field generating device 142 may be implanted in the patient. The electric field generating device 142 may be implanted subcutaneously. Alternatively, the electric field generating device 142 may be positioned outside of the patient's body. For example, the electric field generating device 142 may be attached to the patient's skin or carried by the patient.

Examples of suitable electric electroactive polymers include ferroelectric polymer, dielectric electroactive polymers; electrorestrictive polymers, such as electrorestrictive graft elastomers, electroviscoelastic elastomers, electrostrictive polyurethanes (e.g. electrostrictive carbon black loaded polyurethane); and liquid crystal elastomer materials.

Ferroelectric polymers are a group of crystalline polar polymers that are also ferroelectric (maintain a permanent electric polarization that can be reversed or switched) in an external electric field. Examples of ferroelectric polymers include polyvinylidene fluoride (PVDF) and nylon 11.

Dielectric electroactive polymers are materials in which actuation is caused by electrostatic forces between two electrodes which squeeze the polymer. Dielectric electroactive polymers are discussed in greater detail in WO 2014/086885, incorporated by reference in its entirety.

Electrorestrictive graft elastomers have flexible backbone chains with branching side chains. The side chains on neighboring backbone polymers cross-link and form crystal units which then form polarized monomers which contain atoms with partial charges and generate dipole moments. When an electrical field is applied, a force is applied to each partial charge thereby causing rotation of the whole polymer unit, which causes electrostrictive strain and deformation of the polymer. Electrorestrictive graft elastomers are discussed in greater detail for example in U.S. Pat. No. 6,515,077 incorporated herein in its entirety.

Main-chain liquid crystalline polymers have mesogenic groups linked by a flexible spacer. The mesogens form the mesophase structure that causes the polymer to adopt a conformation compatible with the mesophase structure. Medical devices comprising liquid crystal polymers are described in commonly assigned U.S. Pat. Nos. 8,497,342; 7,662,129; 6,730,377; 7,582,078; 6,284,333; 6,242,063; and 7,101,597, each of which is incorporated by reference in entirety.

The use of electroactive polymers in medical devices are described in commonly assigned U.S. Pat. Nos. 8,398,693; 7,777,399; 8,439,961; 8,414,632; 7,766,896; 8,685,074; 7,909,844; 7,951,186; 8,694,076; and U.S. Patent Application No. 2008/0109061; 2007/0249909; each of which is incorporated by reference in entirety.

### III. Optional Features of the Heart System

The heart system 100 may include one or more areas, bands, coatings, members, etc. that is (are) detectable by imaging modalities such as X-Ray, MRI, ultrasound, etc. In some embodiments at least a portion of the heart system 100 is at least partially radiopaque.

The heart system 100 may be configured to include one or more mechanisms for the delivery of a therapeutic agent. Often the agent will be in the form of a coating or other layer (or layers) of material placed on a surface region of the heart system, which is adapted to be released at the site of the system's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

### IV. Therapeutic Goals of the Heart System

A heart system 100 as disclosed herein is constructed and arranged to accomplish at least one therapeutic goal. Examples of therapeutic goals for a heart system 100 as disclosed herein include: partitioning a dysfunctional heart wall; facilitating the pumping of blood through the heart (facilitate the function of systole); preventing further dilation, enlargement, or remodeling of the heart; changing the volume of the native heart chamber; reducing the volume of blood to be ejected; reducing resistance to blood flow; changing the shape of the native heart chamber; reducing the amount of pressure or stress on the native heart chamber wall; preventing further dilation of the heart chamber wall; providing the heart with an opportunity to recuperate from damage; compensating for stiffness of the native heart chamber wall; treating a heart with impaired function (e.g. heart failure or an enlarged heart); slowing or stopping the progression of damage to the heart (e.g. slow/stop heart failure); and combinations thereof. As discussed below in greater detail, a heart system 100 may be directed to one or more of these therapeutic goals.

The heart system 100 may be further constructed and arranged to prevent or minimize migration of the heart system 100.

### i. Facilitate the Function of Systole

As discussed above, the heart system 100 may improve heart function. In situations where the heart muscle has been damaged, the heart may be unable to pump enough blood to meet the body's needs. Heart failure is an example of a condition where the heart has impaired function because the heart is unable to pump enough blood to meet the body's needs. A heart system 100 constructed and arranged to mechanically facilitate the function of systole helps pump blood to meet the body's needs. Such a heart system 100 may be used treat heart failure; may slow or stop of the progression of heart failure; may prevent dilation, enlargement, or remodeling of the heart; and combinations thereof. The mechanical facilitation of blood flow by the heart system 100 may also provide the heart with an opportunity to recuperate from the damage.

A cooperative mechanism 120 may mechanically facilitate the prosthetic heart chamber 108 in the function of systole by returning to an original shape after diastole and/or providing pressure to the prosthetic heart chamber 108.

For example, the cooperative mechanism 120 may be constructed and arranged to have an original, undeformed configuration with a shape such that the cooperative mechanism impinges or presses on the prosthetic heart chamber 108. Therefore when there is no blood in the prosthetic heart chamber 108 the undeformed cooperative mechanism 120 presses a portion of the prosthetic heart chamber 108 inwards. When the aortic valve is closed blood enters the prosthetic heart chamber 108. As the prosthetic heart chamber 108 fills with blood the cooperative mechanism 120 deforms into a deformed configuration due to the pressure generated in the prosthetic heart chamber 108 by the blood flowing into the prosthetic heart chamber 108. When the aortic valve opens, there is a release of the end diastolic pressure and the cooperative mechanism 120 returns to its original, undeformed, configuration. The return of the cooperative mechanism 120 to its original undeformed configuration provides additional complementary forces along with the native heart wall to contract and eject the blood from the prosthetic heart chamber 108.

Alternatively, the cooperative mechanism 120 may be constructed and arranged to have an original, undeformed configuration during systole and a deformed configuration during diastole. Thus, when blood enters the prosthetic heart chamber 108, the cooperative mechanism 120 will deform outwards. However, when the valve opens to allow blood to be ejected, the cooperative mechanism 120 will return to its original configuration thereby facilitating ejection of the blood from the prosthetic heart chamber 108. Thus, the cooperative mechanism 120 provides a force in addition to the contraction of the native heart to eject blood from the prosthetic heart chamber 108.

Examples of cooperative mechanisms 120 that return to an original shape after diastole and/or provide pressure to the prosthetic heart chamber 108 include filler material 126 (see e.g. FIGS. 8-12); a shape memory element 128 (see e.g. FIGS. 13-15); an expandable member 130 (see e.g. FIGS. 17-19); a cooperative mechanism compartment 112 (see e.g. FIGS. 16 and 20); a pump device 136 (see e.g. FIGS. 16, 18, and 20); a material 134 (see e.g. FIG. 21); and combinations thereof.

A filler material 126 may have an original, undeformed configuration with a shape such that the cooperative mechanism impinges or presses on the prosthetic heart chamber 108 and when the valve opens the filler material 126 returns to its original, undeformed, configuration, thereby providing additional complementary forces along with the native heart wall to contract and eject the blood from the prosthetic heart chamber 108. For example, the filler material 126 may be a foam or a thixotropic material.

A shape memory element 128 may mechanically facilitate the prosthetic heart chamber 108 in the function of systole due to the reversible change in the shape of the shape memory element 128 (see e.g. FIGS. 14-15). For example, when the prosthetic heart chamber 108 fills with blood, a mechanical load is applied by the prosthetic heart chamber 108 onto the shape memory element 128 (see e.g. FIG. 14). The mechanical load may deform the shape memory element 128 from its original, undeformed configuration. In the original, undeformed configuration, the shape memory element 128 is shaped such that it impinges or presses on the prosthetic heart chamber 108 (see e.g. FIG. 15). Therefore when there is no blood in the prosthetic heart chamber the undeformed shape memory element 128 presses a portion of the prosthetic heart chamber 108 inwards. When the aortic valve is closed blood enters the prosthetic heart chamber 108. As the prosthetic heart chamber 108 fills with blood the shape memory element 128 deforms into a deformed configuration due to the pressure generated in the prosthetic heart chamber 108 by the blood flowing into the prosthetic heart chamber 108 (see e.g. FIG. 14). When the aortic valve opens, there is a release of the end diastolic pressure and the shape memory element 128 returns to its original, undeformed, configuration (see e.g. FIG. 15). The return of the shape memory element 128 to its original undeformed configuration provides additional complementary forces along with the native heart wall to contract and eject the blood from the prosthetic heart chamber 108.

As discussed above in section II.A.5-7., a pump device 136 may mechanically facilitate the prosthetic heart chamber 108 in the function of systole by expanding a cooperative mechanism compartment 112 or an expandable member 130 to an enlarged state during systole. When the cooperative mechanism compartment 112 or the expandable member 130 expands to the enlarged state during systole, the volume of the prosthetic heart chamber 108 is reduced thereby moving the blood out of the prosthetic heart chamber 108. The expansion of the expandable cooperative mechanism compartment 112 or expandable member 120 also provides a force against the prosthetic heart chamber 108.

A prosthetic heart chamber 108 comprising a material 134 that is elastic may be combined with anchors 124 that stretch the elastic material 134 taut so that there is space between the prosthetic heart chamber 108 and the native heart wall (see e.g. FIG. 12). Because the material 134 has elasticity, it will be biased to its taut configuration. When blood enters the prosthetic heart chamber 108, the material 134 will stretch outwards. However, when the valve opens to allow blood to be ejected, the elastic material 134 will return to its original taut configuration thereby facilitating ejection of the blood from the prosthetic heart chamber 108. Thus, the elastic material 134 provides a force in addition to the contraction of the native heart to eject blood from the prosthetic heart chamber 108. This arrangement may also provide the heart with an opportunity to recuperate from damage. Recuperation of the heart may prevent the onset of heart failure; may slow the progression of damage that may lead to heart failure; or may slow the progression of heart failure.

As discussed above in section II.A.8., a prosthetic heart chamber 108 comprising an electric electroactive polymer material 134 may facilitate the prosthetic heart chamber 108 in the function of systole. For example, during systole, an electric field is applied so that the electric electroactive polymer material exhibits a change in size and/or shape, thereby reducing the volume of the prosthetic heart chamber 108 in order to move blood out of the prosthetic heart chamber 108.

### ii. Separating/Partitioning an Inner Volume of Native Heart Chamber

Separating or partitioning an inner volume of the native heart chamber from an area of dysfunctional native heart wall may prevent further dilation of the dysfunctional native heart wall and/or prevent or slow further progression of heart failure. Without being bound by theory, providing a separation or empty space between the prosthetic heart chamber 108 and the wall 8 reduces the stress on that portion of the wall 8. Thus, partitioning may provide the heart with an opportunity to recuperate from, or slow the progression of, heart damage. Recuperation of the heart may prevent the onset of heart failure; may slow the progression of heart damage that may lead to heart failure; or may slow the progression of heart failure.

Examples of cooperative mechanisms 120 that may be used to separate/partition an area of dysfunctional native heart wall include: an adhesive 122; an anchor 124; filler material 126; and combinations thereof.

One exemplary way to separate/partition an area of dysfunctional native heart wall is implanting the heart system 100 so that a portion of the prosthetic heart chamber 108 is stretched taut so that there is an empty partition space between the outer surface of the prosthetic heart chamber 108 and the wall 8 of the native heart chamber (see e.g. FIGS. 7, 12, 14-15, and 17). Adhesive 122 and/or anchors 124 may be used to maintain a portion of the prosthetic heart chamber 108 taut and/or to maintain an empty space between the prosthetic heart chamber 108 and the native heart wall.

Another exemplary way to partition an area of dysfunctional native heart wall is to provide filler material 126 between the native heart wall and the prosthetic heart chamber 108.

### iii. Reduce Volume of Blood to Be Ejected

The heart system 100 may be constructed and arranged to reduce the volume of blood to be ejected from the prosthetic heart chamber 108 during systole. The modification of the shape and/or volume of the prosthetic heart chamber 108 may occur during diastole; during systole; or during both diastole and systole.

To reduce the volume of blood to be ejected, the prosthetic heart chamber 108 may have a size less than the size of the native heart chamber.

The shape of the prosthetic heart chamber 108 may be modified to effect a reduction in the volume of the main lumen 110 of the prosthetic heart chamber 108. A modification of the shape and/or volume of the prosthetic heart chamber 108 in diastole may reduce the volume of blood to be ejected during systole. Reducing the volume of blood ejected during systole may reduce pressure on the wall 8 of the native heart chamber; may define a capacity that can be efficiently ejected from the native heart chamber; and combinations thereof. Reducing the amount of pressure on the native heart wall may allow the heart to recuperate from, or slow the progression of, heart damage. Recuperation of the heart may prevent the onset of heart failure; may slow the progression of heart damage that may lead to heart failure; or may slow the progression of heart failure.

The heart system 100 may have prosthetic heart chamber 108 with a size equal to the size of the native heart chamber may be modified by a cooperative mechanism 120 to have a size less than the size of the native heart chamber. Cooperative mechanisms 120 that may be used to reduce the volume of blood to be ejected from the prosthetic heart chamber 108 include: an adhesive 122; an anchor 124, filler material 126; an expandable member 130; cooperative mechanism compartment 112; pump device 136; material 134; and combinations thereof.

Positioning a cooperative mechanism 120 between the prosthetic heart chamber 108 and the native heart chamber wall 8 is one way to reduce the volume of the prosthetic heart chamber 108 with a size equal to the size of the native heart chamber.

Separating or partitioning an inner volume of the native heart chamber with anchors 124 as discussed above may also modify the shape and/or volume of the prosthetic heart chamber 108.

Filler material 126 may reduce the volume of blood to be ejected from the prosthetic heart chamber 108. The filler material 126 may reduce the cross-sectional area of a portion of the prosthetic heart chamber 108. This reduces the amount or volume of blood passing through that portion of the prosthetic heart chamber 108. This impacts the pressure gradient in that region and/or impacts the resistance to blood flow, which impacts the amount of pressure on the native heart wall. Thus, for example more filler material 126 may be positioned in the region between the bottom of the native heart chamber and the prosthetic heart chamber 108. This reduces the volume of blood in the bottom region of the prosthetic heart chamber 108. This is beneficial if the native heart wall is dysfunctional in this region and it is difficult for the native heart to pump blood from the bottom region.

The expandable cooperative mechanism compartment 112 may also reduce the volume of blood to be ejected from the prosthetic heart chamber 108. For example, the size of the main lumen 110 of the prosthetic heart chamber 108 in diastole may be reduced by increasing the size of the expandable cooperative mechanism compartment 112. Further increasing the size of the expandable cooperative mechanism compartment 112 during systole will mechanically facilitate the prosthetic heart chamber 108 in the function of systole.

An expandable member 130 may also be constructed and arranged to reduce the volume of blood to be ejected from the prosthetic heart chamber 108. For example, the size of the main lumen 110 of the prosthetic heart chamber 108 in diastole may be reduced by increasing the size of the expandable member 130. Further increasing the size of the expandable member 130 during systole will mechanically facilitate the prosthetic heart chamber 108 in the function of systole.

A pump device 136 may also be used to reduce the volume of blood to be ejected from the prosthetic heart chamber 108. Without being bound by theory, reducing the volume of blood to be ejected from the prosthetic heart chamber 108 may reduce the pressure/stress on the native heart chamber wall 8. Reducing the amount of pressure or stress on the native heart chamber wall 8 may provide the heart with an opportunity to recuperate from damage. Recuperation of the heart may prevent the onset of heart failure; may slow the progression of damage that may lead to heart failure; or may slow the progression of heart failure.

### iv. Reduce Resistance to Blood Flow

The heart system 100 may be constructed and arranged to reduce resistance to blood flow through the prosthetic heart chamber 108. The cooperative mechanism 120 may reduce the cross-sectional area of a portion of the prosthetic heart chamber 108. Reducing the cross-sectional area impacts the pressure gradient in that region which impacts the resistance to blood flow. Without being bound by theory, reducing the resistance to blood flow may also reduce the amount of pressure or stress on the native heart chamber wall 8. Reducing the amount of pressure or stress on the native heart chamber wall 8 may provide the heart with an opportunity to recuperate from damage. Recuperation of the heart may prevent the onset of heart failure; may slow the progression of damage that may lead to heart failure; or may slow the progression of heart failure.

Cooperative mechanisms 120 that may be used to reduce the cross-sectional sectional area of a portion of the prosthetic heart chamber 108 include: adhesive 122; an anchor 124; filler material 126; a cooperative mechanism compartment 112; an expandable member 130; a pump device 136; a material 134; and combinations thereof.

For example, as discussed above, a cooperative mechanism 120 may reduce the volume or modify the shape of the prosthetic heart chamber 108. This may also reduce the cross-sectional area of a portion of the prosthetic heart chamber 108.

### v. Compensate for Stiffness of Native Heart Wall

The cooperative mechanism 120 may compensate for stiffness (e.g. myocardial stiffness). Stiffness may be due to an increased deposition of collagen. A normally functioning heart relaxes and expands during diastole and contracts during systole. Stiffness affects the flexibility of the heart muscle. Thus, the ability of the heart to relax and expand during diastole, and to contract during systole is affected. When the heart does not relax normally during diastole, the amount of blood that enters the native heart chamber may be reduced. Stiffness of the heart also affects the amount of blood that is ejected from the native heart chamber.

Cooperative mechanisms 120 that aid in the ejection of blood from the native heart chamber may be used to compensate for stiffness of the native heart chamber. Thus, for example cooperative mechanisms 120 as discussed above that facilitate the function of systole may compensate for stiffness of the native heart chamber.

Cooperative mechanisms 120 that may be used to compensate for stiffness of the heart chamber include: adhesive 122; an anchor 124; a shape memory element 128; a cooperative mechanism compartment 112; an expandable member 130; a pump device 136; a material 134; and combinations thereof.

### vi. Preventing/Minimizing Migration of the Heart System

As discussed above, the heart system 100 may be constructed and arranged to prevent or minimize migration of the heart system 100. A cooperative mechanism 120 may prevent or minimize migration of the prosthetic heart chamber assembly 102 by securing the prosthetic heart chamber assembly 102 to the wall 8 of the native heart chamber. Maintaining the location of the heart system 100 in the native heart chamber may help ensure that the therapeutic goals of the heart system 100 are obtained.

Examples of cooperative mechanisms 120 that secure the prosthetic heart chamber assembly 102 to the wall 8 of the native heart chamber include: an adhesive 122 (see e.g. FIGS. 5 and 19); an anchor 124 (see e.g. FIGS. 6-7, 12, 14-15, and 17-18); and combinations thereof. The prosthetic heart chamber assembly 102 may be secured to a single location of wall 8 of the native heart chamber (see e.g. FIG. 6), or to a plurality of locations (see e.g. FIGS. 7, 12, 14-15, and 17-18).

A heart system 100 directed to one or more of the therapeutic goals discussed above may also be constructed and arranged to prevent/minimize migration of the heart system 100 due to the use of adhesive 122 and/or anchors 124.

### EXAMPLES

Exemplifications of a prosthetic heart chamber assembly 102 and a heart system 100 as described above are provided by the following non-limiting examples.

### Example 1

A heart system 100 comprises a prosthetic heart chamber assembly 102 and a cooperative mechanism 120.

The prosthetic heart chamber assembly 102 includes a first prosthesis 104, a second prosthesis 106, and a prosthetic heart chamber 108.

The prostheses 104, 106 of the heart system 100 may further include anchoring means.

The cooperative mechanism 120 may be selected from the group consisting of: adhesive 122; an anchor 124; a filler material 126; a shape memory element 128; a pump device 136; a cooperative mechanism compartment 112; an expandable member 130; a material 134 attached to, or forming a part of, the wall of the prosthetic heart chamber 108; and combinations thereof.

### Example 2

The heart system 100 of Example 1 may be constructed and arranged to prevent or minimize migration of the heart system 100.

The cooperative mechanism 120 may be selected from the group consisting of: adhesive 122, an anchor 124, and combinations thereof.

The prostheses 104, 106 of the heart system 100 may further include anchoring means.

25

### Example 3

The heart system 100 of any one of Examples 1 or 2, wherein the heart system 100 is constructed and arranged for a therapeutic goal selected from the group consisting of: partitioning a dysfunctional heart wall; facilitate the pumping of blood through the heart (facilitate the function of systole); preventing further dilation, enlargement, or remodeling of the heart; changing the volume of the native heart chamber; reducing the volume of blood to be ejected; reducing resistance to blood flow; changing the shape of the native heart chamber; reducing the amount of pressure or stress on the native heart chamber wall; preventing further dilation of the heart chamber wall; providing the heart with an opportunity to recuperate from damage; compensating for stiffness of the native heart chamber wall; treating a damaged heart (e.g. heart failure); slowing or stopping the progression of damage to the heart (e.g. slow/stop heart failure); and combinations thereof.

### Example 4

The heart system 100 of any one of Examples 1, 2, or 3, wherein the heart system 100 is constructed and arranged to facilitate the pumping of blood through the heart (facilitate the function of systole).

Cooperative mechanisms 120 for a heart system 100 constructed and arranged to facilitate the pumping of blood through the heart (facilitate the function of systole) may be selected from the group consisting of: filler material 126, a shape memory element 128, a pump device 136; a cooperative mechanism compartment 112; an expandable member 130; a material 134; and combinations thereof.

### Example 5

The heart system 100 of any one of Examples 1, 2, 3, or 4, wherein the heart system 100 has a cooperative mechanism 120 that is constructed and arranged to return to an original shape after diastole.

Cooperative mechanisms 120 for a heart system 100 constructed and arranged to return to an original shape after diastole may be selected from the group consisting of filler material 126; a shape memory element 128; an expandable member 130; cooperative mechanism compartment 112; a pump device 136; a material 134; and combinations thereof.

### Example 6

The heart system 100 of any one of Examples 1, 2, 3, 4, or 5, wherein the heart system 100 has a cooperative mechanism 120 that is constructed and arranged to provide pressure to, or impart a force to, the prosthetic heart chamber 108.

Cooperative mechanisms 120 for a heart system 100 constructed and arranged to provide pressure to, or impart a force to, the prosthetic heart chamber 108 may be selected from the group consisting of: filler material 126; a shape memory element 128; an expandable member 130; a cooperative mechanism compartment 112; a pump device 136; a material 134; and combinations thereof.

### Example 7

The heart system 100 of any one of Examples 1, 2, 3, 4, 5, 6, 7, 8, 9, 10, 11, 12, or 13, wherein the heart system 100 is constructed and arranged to change the volume of the native heart chamber.

Cooperative mechanisms 120 for a heart system 100 constructed and arranged to change the volume of the native heart chamber may be selected from the group consisting of: adhesive 122; anchors 124; filler material 126; a pump device 136; an expandable member 130, a cooperative mechanism compartment 112; a material 134; and combinations thereof.

### Example 8

The heart system 100 of any one of Examples 1, 2, 3, or 4, wherein the heart system 100 has a cooperative mechanism 120 that is constructed and arranged to reduce the volume of blood to be ejected.

Cooperative mechanisms 120 for a heart system 100 constructed and arranged to reduce the volume of blood to be ejected may be selected from the group consisting of: adhesive 122; an anchor 124; filler material 126; a pump device 136; an expandable member 130, a cooperative mechanism compartment 112; a material 134; and combinations thereof.

### Example 9

The heart system 100 of any one of Examples 1, 2, 3, 4, 5, or 6, wherein the heart system 100 is constructed and arranged to change the shape of the native heart chamber.

Cooperative mechanisms 120 for a heart system 100 constructed and arranged to change the shape of the native heart chamber may be selected from the group consisting of adhesive 122; an anchor 124; filler material 126; a shape memory element 128; a pump device 136; an expandable member 130; a cooperative mechanism compartment 112; a material 134; and combinations thereof.

### Example 10

The heart system 100 of any one of Examples 1, 2, 3, 4, 5, 6, or 7, wherein the heart system 100 is constructed and arranged to reduce the amount of pressure or stress on the native heart chamber wall.

Cooperative mechanisms 120 for a heart system 100 constructed and arranged to reduce the amount of pressure or stress on the native heart chamber wall may be selected from the group consisting of: adhesive 122; an anchor 124; filler material 126; a cooperative mechanism compartment 112; an expandable member 130; a pump device 136; a material 134; and combinations thereof.

### Example 11

The heart system 100 of any one of Examples 1, 2, 3, 4, 5, 6, 7, or 8, wherein the heart system 100 is constructed and arranged to prevent further dilation, enlargement, or remodeling of the native heart chamber wall.

Cooperative mechanisms 120 for a heart system 100 constructed and arranged to prevent further dilation of the heart chamber wall may be selected from the group consisting of: adhesive 122; an anchor 124; filler material 126; and combinations thereof.

### Example 12

The heart system 100 of any one of Examples 1, 2, 3, 4, 5, 6, 7, 8, or 9, wherein the heart system 100 is constructed and arranged to reduce resistance to blood flow.

Cooperative mechanisms 120 for a heart system 100 constructed and arranged to reduce resistance to blood flow may be selected from the group consisting of: adhesive 122; an anchor 124; filler material 126; a cooperative mechanism compartment; an expandable member 130; a pump device 136; a material 134; and combinations thereof.

### Example 13

The heart system 100 of any one of Examples 1, 2, 3, 4, 5, 6, 7, 8, 9, or 10, wherein the heart system 100 is constructed and arranged to partition a damaged section of the native heart chamber wall.

Cooperative mechanisms 120 for a heart system 100 constructed and arranged to partition a damaged section of the native heart chamber wall may be selected from the group consisting of: adhesive 122; an anchor 124; filler material 126; a material 134; and combinations thereof.

### Example 14

The heart system 100 of any one of Examples 1, 2, 3, 4, 5, 6, 7, 8, 9, 10, or 11, wherein the heart system 100 is constructed and arranged to compensate for stiffness of the native heart chamber wall.

Cooperative mechanisms 120 for a heart system 100 constructed and arranged to compensate for stiffness of the native heart chamber wall may be selected from the group consisting of: adhesive 122; an anchor 124; filler material 126; a shape memory element 128; a pump device 136; a cooperative mechanism compartment 112; an expandable member 130; a material 134; and combinations thereof.

### Example 15

The heart system 100 of any one of Examples 1, 2, 3, 4, 5, 6, 7, 8, 9, 10, 11, 12, or 13, wherein the heart system 100 is constructed and arranged to provide the heart with an opportunity to recuperate from damage.

Cooperative mechanisms 120 for a heart system 100 constructed and arranged to provide the heart with an opportunity to recuperate from damage may be selected from the group consisting of: adhesive 122; an anchor 124; filler material 126; a shape memory element 128; a pump device 136; a cooperative mechanism compartment 112; an expandable member 130; a material 134; and combinations thereof.

### Example 16

The heart system 100 of any one of Examples 1, 2, 3, 4, 5, 6, 7, 8, 9, 10, 11, 12, 13, or 14, wherein the heart system 100 is constructed and arranged to treat a damaged heart (e.g. heart failure).

Cooperative mechanisms 120 for a heart system 100 constructed and arranged to treat a damaged heart (e.g. heart failure) may be selected from the group consisting of: adhesive 122; an anchor 124; filler material 126; a shape memory element 128; a pump device 136; a cooperative mechanism compartment 112; an expandable member 130; a material 134; and combinations thereof.

### Example 17

The heart system 100 of any one of Examples 1, 2, 3, 4, 5, 6, 7, 8, 9, 10, 11, 12, 13, 14, or 15, wherein the heart system 100 is constructed and arranged to slow or stop the progression of damage to the heart (e.g. slow/stop heart failure).

Cooperative mechanisms 120 for a heart system 100 constructed and arranged to slow or stop the progression of damage to the heart (e.g. slow/stop heart failure) may be selected from the group consisting of: adhesive 122; an anchor 124; filler material 126; a shape memory element 128; a pump device 136; a cooperative mechanism compartment 112; an expandable member 130; a material 134; and combinations thereof.

### Example 18

A prosthetic heart chamber assembly 102 suitable for a heart system 100 as described in any one of Examples 1-16 may comprise a first prosthesis 104 that is a valve and a second prosthesis 106that is a valve (see e.g. FIG. 3A).

### Example 19

A prosthetic heart chamber assembly 102 suitable for a heart system 100 as described in any one of Examples 1-16 may comprise a first prosthesis 104 that is a tubular prosthesis and a second prosthesis 106 that is a valve.

The tubular prosthesis may be selected from the group consisting of a stent, a stent-graft, and a graft.

### Example 20

The prosthetic heart chamber assembly 102 may further comprise a cooperative mechanism compartment 112. The cooperative mechanism compartment 112 defines a compartment lumen 111 separate from the main lumen 110.

The cooperative mechanism compartment 112 of the prosthetic heart chamber assembly 102 may have a cooperative mechanism 120 positioned therein. The cooperative mechanism 120 may be selected from the group consisting of a filler material 126; a shape memory element 128; and an expandable member 130.

The cooperative mechanism compartment 112 may be the cooperative mechanism 120. The cooperative mechanism compartment 112 may be expandable; may be formed of a material 134; and combinations thereof.

### Example 21

A heart system 100 comprises a prosthetic heart chamber assembly 102 and an adhesive 122 (see e.g. FIG. 5).

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The adhesive 122 is the cooperative mechanism 120 for heart system 100.

### Example 22

FIG. 6 shows an example of a heart system 100. The heart system 100 has a prosthetic heart chamber assembly 102 and an anchor 124.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The anchor 124 is the cooperative mechanism 120 for the heart system 100. The anchor 124 is a single anchor 124. The anchor 124 has an end region that spirals. When the heart system 100 is implanted, the end of the anchor 124 may be positioned in the wall the native heart chamber. When the heart system 100 is implanted, the anchor 124 may prevent/minimize migration of the heart system 100.

### Example 23

FIG. 7 shows an example of a heart system 100 as described above. The system 100 has a prosthetic heart chamber assembly 102; and anchors 124.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The anchors 124 may include a first anchor and a second anchor. Each anchor 124 is a cooperative mechanism 120.

### Example 24

FIG. 8 shows an example of a heart system 100 as described above. The system 100 has a prosthetic heart chamber assembly 102; and a filler material 126.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The filler material 126 is the cooperative mechanism 120 of the heart system 100.

When the heart system 100 is implanted, the filler material 126 may surround the prosthetic heart chamber 108.

When the heart system 100 is implanted, the filler material 126 may modify the shape of the prosthetic heart chamber 108.

When the heart system 100 is implanted, the filler material 126 may modify the size of the prosthetic heart chamber 108.

### Example 25

FIG. 12 shows an example of a heart system 100 as described above. The heart system 100 has a prosthetic heart chamber assembly 102; anchors 124; and a filler material 126.

The prosthetic heart chamber assembly 102 comprises a first prosthesis 104, a second prosthesis 106, and a prosthetic heart chamber 108.

The prosthetic heart chamber 108 defines a main lumen 110.

The first prosthesis 104 may be a valve. The second prosthesis may be a valve or a tubular prosthesis. The tubular prosthesis may be selected from the group consisting of a stent, a stent-graft, and a graft.

The anchors 124 and the filler material 126 are cooperative mechanisms 120 of the heart system 100.

The filler material 126 may only partially surround the prosthetic heart chamber 108.

When the heart system 100 is implanted the portion of the wall 8 of the native heart chamber defining the area with no filler material between the prosthetic heart chamber 108 and the wall 8 of the native heart chamber may be dysfunctional due e.g. to a myocardial infarction.

### Example 26

The heart system 100 has a prosthetic heart chamber assembly 102, an adhesive 122, and a filler material 126.

The prosthetic heart chamber assembly 102 comprises a first prosthesis 104, a second prosthesis 106, and a prosthetic heart chamber 108.

The prosthetic heart chamber 108 defines a main lumen 110.

The first prosthesis 104 may be a valve. The second prosthesis may be a valve or a tubular prosthesis. The tubular prosthesis may be selected from the group consisting of a stent, a stent-graft, and a graft.

The adhesive 122 and the filler material 126 are cooperative mechanisms 120 of the heart system 100.

The filler material 126 may only partially surround the prosthetic heart chamber 108. The adhesive 122 may enclose an area to be free of filler material 126.

When the heart system 100 is implanted the portion of the wall 8 of the native heart chamber defining the area with no filler material between the prosthetic heart chamber 108 and the wall 8 of the native heart chamber may be dysfunctional due e.g. to a myocardial infarction.

### Example 27

A heart system 100 has a prosthetic heart chamber assembly 102 and a shape memory element 128.

The prosthetic heart chamber assembly 102 comprises a first prosthesis 104, a second prosthesis 106, and a prosthetic heart chamber 108.

The prosthetic heart chamber 108 defines a main lumen 110.

The first prosthesis 104 may be a valve. The second prosthesis may be a valve or a tubular prosthesis. The tubular prosthesis may be selected from the group consisting of a stent, a stent-graft, and a graft.

The shape memory element is the cooperative mechanism 112 of the heart system 100.

The heart system 100 may have a single shape memory element 128, or a plurality of shape memory elements 128.

The shape memory element 128 may comprise a shape memory alloy with superelasticity or a shape memory polymer.

The shape memory element 128 has an atraumatic shape. The shape memory element 128 may have end regions that are curled or curved

When the heart system 100 is implanted, the shape memory element 128 may be free floating between the prosthetic heart chamber assembly 102 and the wall 8 of the native heart chamber; may be secured to the prosthetic heart chamber assembly 102; may form a part of the prosthetic heart chamber 108; or positioned within a cooperative mechanism compartment 112 of the prosthetic heart chamber 108 of the prosthetic heart chamber assembly 102.

### Example 28

A heart system 100 has a prosthetic heart chamber assembly 102, anchors 124; and a shape memory element 128.

The prosthetic heart chamber assembly 102 comprises a first prosthesis 104, a second prosthesis 106, and a prosthetic heart chamber 108.

The prosthetic heart chamber 108 defines a main lumen 110.

The first prosthesis 104 may be a valve. The second prosthesis may be a valve or a tubular prosthesis. The tubular prosthesis may be selected from the group consisting of a stent, a stent-graft, and a graft.

The anchors 124 and the shape memory element 128 are the cooperative mechanism 112 of the heart system 100.

The shape memory element 128 may comprise a shape memory alloy with superelasticity or a shape memory polymer.

The shape memory element 128 has an atraumatic shape. The shape memory element 128 may have end regions that are curled or curved

When the heart system 100 is implanted the shape memory element 128 is positioned in an area defined by the prosthetic heart chamber 108 and the wall 8 of the native heart chamber.

### Example 29

FIGS. 16A-B and 20 show an example of a heart system 100 as described above. The heart system 100 has a prosthetic heart chamber assembly 102 comprising an expandable cooperative mechanism compartment 112, where the expandable cooperative mechanism compartment 112 is in fluid communication with a pump device 136.

The prosthetic heart chamber assembly 102 comprises a first prosthesis 104, a second prosthesis 106, and a prosthetic heart chamber 108.

The prosthetic heart chamber 108 defines a main lumen 110.

The first prosthesis 104 may be a valve. The second prosthesis may be a valve or a tubular prosthesis. The tubular prosthesis may be selected from the group consisting of a stent, a stent-graft, and a graft.

The cooperative mechanism compartment 112 is the cooperative mechanism 120 of the heart system 100.

The pump device 136 adds and withdraws expansion media from the cooperative mechanism compartment 112.

When the heart system 100 is implanted, the size of the cooperative mechanism compartment 112 increases during systole and decreases during diastole.

### Example 30

A heart system 100 has a prosthetic heart chamber assembly 102 comprising a prosthetic heart chamber; and a material 134 attached to, or forming a part of, the wall of the prosthetic heart chamber.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The material 134 attached to, or forming a part of, the wall of the prosthetic heart chamber may comprise elastic material.

The material 134 forming a part of the wall of the prosthetic heart chamber 108 may comprise a section of elastic material and a section of non-elastic material.

The material 134 attached to, or forming a part of, the wall of the prosthetic heart chamber may comprise an electric electroactive polymer. The electric electroactive polymer may be in one or more sections. An electric field generating device 142 is associated with the prosthetic heart chamber assembly 102 (see e.g. FIG. 21).

When the heart system 100 is implanted, the size of the prosthetic heart chamber 108 increases during systole and decreases during diastole.

### Example 31

FIG. 17 shows an example of a heart system 100 as described above. The heart system 100 has a prosthetic heart chamber assembly 102; anchors 124; and an expandable member 130.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The anchors 124 and the expandable member 130 are the cooperative mechanisms 120 of the heart system 100.

When the heart system 100 is implanted the expandable member 130 is positioned in an area defined by the prosthetic heart chamber 108 and the wall 8 of the native heart chamber.

### Example 32

FIGS. 18A-B show an example of a heart system 100 as described above. The heart system 100 has a prosthetic heart chamber assembly 102; anchors 124; an expandable member 130; and a pump device 136.

The prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis may be a valve or a tubular prosthesis. The tubular prosthesis may be selected from the group consisting of a stent, a stent-graft, and a graft.

The anchors 124, the expandable member 130, and the pump device 136 are the cooperative mechanisms 120 of the heart system 100.

When the heart system 100 is implanted the expandable member 130 is positioned in an area defined by the prosthetic heart chamber 108 and the wall 8 of the native heart chamber.

When the heart system 100 is implanted, the size of the expandable member 130 increases during systole and decreases during diastole.

### Methods

A heart system 100 as disclosed herein may be delivered by a delivery system suitable for transcatheter aortic valve implantation (TAVI). Commonly assigned U.S. U2014/0277408, incorporated by herein in its entirety, discloses a delivery system that may be used to deliver a heart system 100 as disclosed herein. The prosthetic heart chamber assembly 102 and the cooperative mechanism 120 may be implanted simultaneously or sequentially. The delivery system is not a “cooperative mechanism” as disclosed herein.

### Method 1

A method to treat a heart with impaired function comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a shape memory element 128; a pump device 136; a cooperative mechanism compartment 112; an expandable member 130; a material 134; and combinations thereof.

### Method 2

A method to facilitate the pumping of blood through the heart (facilitate the function of systole) comprises: implanting a heart system 100 comprising a prosthetic heart chamber 102 and a cooperative mechanism 120.

The cooperative mechanisms 120 may be selected from the group consisting of: filler material 126, a shape memory element 128, a pump device 136; a cooperative mechanism compartment 112; an expandable member 130; a material 134; and combinations thereof.

### Method 3

The method of Method 2 wherein the cooperative mechanism 120 is constructed and arranged to return to an original shape after diastole.

The cooperative mechanisms 120 may be selected from the group consisting of: filler material 126; a shape memory element 128; an expandable member 130; a cooperative mechanism compartment 112; a pump device 136; a material 134; and combinations thereof.

### Method 4

The method of Methods 2 or 3, wherein the cooperative mechanism 120 is constructed and arranged to provide pressure to, or impart a force to, the prosthetic heart chamber 108.

The cooperative mechanisms 120 may be selected from the group consisting of: filler material 126; a shape memory element 128; an expandable member 130; a cooperative mechanism compartment 112; a pump device 136; a material 134; and combinations thereof.

### Method 5

A method to change the volume of the native heart chamber comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a pump device 136; an expandable member 130, a cooperative mechanism compartment 112; a material 134; and combinations thereof.

### Method 6

A method to reduce the volume of blood to be ejected during systole comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a pump device 136; an expandable member 130, a cooperative mechanism compartment 112; a material 134; and combinations thereof.

### Method 7

A method to change the shape of the native heart chamber comprises: implanting a heart system 100 comprising a prosthetic heart chamber 102 and a cooperative mechanism 120.

The cooperative mechanisms 120 may be selected from the group consisting of adhesive 122; an anchor 124; filler material 126; a shape memory element 128; a pump device 136; an expandable member 130; a cooperative mechanism compartment 112; a material 134; and combinations thereof.

### Method 8

A method to reduce the amount of pressure or stress on the native heart chamber wall comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a shape memory element 128; a pump device 136; a cooperative mechanism compartment 112; an expandable member 130; a material 134; and combinations thereof.

### Method 9

A method to prevent further dilation, enlargement or remodeling, of the native heart chamber wall comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; and combinations thereof.

### Method 10

A method to reduce resistance to blood flow comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a pump device 136; a cooperative mechanism compartment; an expandable member 130; a material 134; and combinations thereof.

### Method 11

A method to partition a damaged section of the native heart chamber wall comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a material 134; and combinations thereof.

### Method 12

A method to compensate for stiffness of the native heart chamber wall comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a shape memory element 128; a pump device 136; a cooperative mechanism compartment 112; an expandable member 130; a material 134; and combinations thereof.

### Method 13

A method to provide the heart with an opportunity to recuperate from damage comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a shape memory element 128; a pump device 134; a cooperative mechanism compartment 112; an expandable member 130; a material 134; and combinations thereof.

### Method 15

A method to treat a damaged heart (e.g. heart failure) comprises: implanting a heart system 100 comprising a prosthetic heart chamber 102 and a cooperative mechanism 120.

The cooperative mechanisms 120 may be selected from the group consisting of: adhesive 122; an anchor 124; filler material 126; a shape memory element 128; a pump device 136; a cooperative mechanism compartment 112; an expandable member 130; a material 134; and combinations thereof.

### Method 16

A method of forming a heart system comprises: providing a heart chamber assembly 102 and a cooperative mechanism 120.

The cooperative mechanism 120 may be selected from the group consisting of: adhesive 122; an anchor 124; filler material 126; a shape memory element 128; a pump device 136; a cooperative mechanism compartment 112; an expandable member 130; a material 134; and combinations thereof.

Wherein the prosthetic heart chamber assembly 102 comprises a first prosthesis 104, a second prosthesis 106, and a prosthetic heart chamber 108. The prosthetic heart chamber 108 defines a main lumen 110. The first prosthesis 104 may be a valve. The second prosthesis 106 may be a valve or a tubular prosthesis. The tubular prosthesis may be selected from the group consisting of a stent, a stent-graft, and a graft.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. An implantable heart system comprising:
a prosthetic heart chamber assembly comprising a first prosthesis, a second prosthesis, and a prosthetic heart chamber interconnecting the first and second prostheses;
a cooperative mechanism associated with the prosthetic heart chamber assembly,
wherein the heart system is constructed and arranged to treat a heart with impaired function.

2. The implantable heart system of claim 1, wherein the first prosthesis is selected from the group consisting of a stent, a stent-graft, a graft, and a valve; and the second prosthesis is a valve.

3. The implantable heart system of claim 1, wherein the cooperative mechanism is constructed and arranged to anchor the prosthetic heart chamber.

4. The implantable heart system of claim 1, wherein the cooperative mechanism is a plurality of cooperative mechanisms.

5. The implantable heart system of claim 1, wherein the cooperative mechanism is secured to the prosthetic heart chamber assembly.

6. The implantable heart system of claim 1, wherein the cooperative mechanism is positioned inside a cooperative mechanism compartment of the prosthetic heart chamber.

7. The implantable heart system of claim 1, wherein the cooperative mechanism is selected from the group consisting of an adhesive; an anchor; a filler material; a shape memory element; an expandable member; a pumping agent; material attached to, or forming a part of, the wall of the prosthetic heart chamber; and combinations thereof.

8. The implantable heart system of claim 7, wherein the material attached to, or forming a part of, the wall of the prosthetic heart chamber is selected from the group consisting of an elastic polymer; an electric electroactive polymer; and combinations thereof.

9. The implantable heart system of claim 8, wherein the cooperative mechanism is the material of the prosthetic heart chamber.

10. The implantable heart system of claim 1, wherein the cooperative mechanism is an anchor.

11. The implantable heart system of claim 1, wherein the cooperative mechanism is a shape memory element.

12. The implantable heart system of claim 1, wherein the cooperative mechanism is an expandable member.

13. The implantable heart system of claim 1, wherein the cooperative mechanism comprises a first cooperative mechanism and a second cooperative mechanism different than the first cooperative mechanism.

14. The implantable heart system of claim 13, wherein the first cooperative mechanism is constructed and arranged to anchor the prosthetic heart chamber.

15. The implantable heart system of claim 13, wherein the first cooperative mechanism is an adhesive and the second cooperative mechanism is a filler material.

16. A prosthetic heart chamber assembly for use with an implantable heart system, the prosthetic heart chamber assembly comprising a valve, a tubular prosthesis, and a prosthetic heart chamber interconnecting the valve and the tubular prosthesis.

17. The prosthetic heart chamber assembly of claim 16, forming a part of an implantable heart system, the heart system further comprising a cooperative mechanism.

18. The prosthetic heart chamber assembly of claim 17, wherein the cooperative mechanism is selected from the group consisting of an adhesive; an anchor; a filler material; a shape memory element; an expandable member; a pumping agent; material attached to, or forming a part of, the wall of the prosthetic heart chamber; and combinations thereof.

19. A heart system constructed and arranged to isolate a damaged section of a native heart chamber wall, the heart system comprising a prosthetic heart chamber and a cooperative element, wherein the cooperative element and the prosthetic heart chamber cooperative to form a space between the prosthetic heart chamber and the native heart chamber wall when the heart system is implanted in a heart chamber.

20. The heart system of claim 19, wherein the cooperative element is selected from the group consisting of adhesives; anchors; material attached to, or forming a part of, a wall of the prosthetic heart chamber; and combinations thereof.
